(19) United States
(12) Patent Application Publication
(10) Pub. No.: US 2024/0091026 A1
(43) Pub. Date: Mar. 21, 2024

(54) SACROILIAC FUSION SYSTEMS AND METHODS

(71) Applicant: SAIL Fusion, LLC, Del Mar, CA (US)

(72) Inventors: Benjamin Arnold, Del Mar, CA (US); Ivet Avalos, Del Mar, CA (US); Brian Bowman, Del Mar, CA (US); David Jansen, Del Mar, CA (US)

(21) Appl. No.: 18/368,658

(22) Filed: Sep. 15, 2023

Related U.S. Application Data
(60) Provisional application No. 63/407,064, filed on Sep. 15, 2022.

(51) Int. Cl. A61F 2/46; A61F 2/30

(52) U.S. Cl. CPC A61F 2/4603; A61F 2/30749; A61F 2002/2835; A61F 2002/30784; A61F 2002/3093; A61F 2002/30995

(57) ABSTRACT

Devices and methods for fusing a sacroiliac joint of a patient. Exemplary fusion systems include an implant body and one or more screws. An implant body can have a medial side configured to engage a sacrum of a patient, a lateral side configured to engage an ilium of the patient, a distal portion, and a proximal portion having an opening configured to receive a screw. A screw can include a distal portion having a threaded portion configured to engage bone of the patient, and a proximal portion configured engage the opening in the proximal portion of the implant body.

### CROSS-REFERENCES TO RELATED APPLICATIONS

This application claims the benefit of U.S. Provisional Patent Application No. 63/407,064 filed Sep. 15, 2022, the disclosure of which is incorporated herein by reference.

### BACKGROUND

Embodiments of the present invention relate generally to surgical instruments and procedures, and in particular embodiments, to sacroiliac fusion systems and methods.

Surgeons may follow any of a variety of procedures in order to treat the joint of a patient. Although existing procedures can provide beneficial results for patients, still further improvements are desired. Embodiments of the present invention provide solutions to at least some of these outstanding needs.

### SUMMARY

The present disclosure generally relates to medical devices and processes and more particularly relates to sacroiliac fusion instruments and procedures.

In one aspect, embodiments of the present invention encompass a system having an interbody and a screw.

In another aspect, embodiments of the present invention encompass sacroiliac fusion systems for treating a patient. Exemplary systems can include an implant body having a medial side configured to engage a sacrum of a patient, a lateral side configured to engage an ilium of the patient, a distal portion, and a proximal portion having an opening configured to receive a screw. Systems can also include a screw having a distal portion and proximal portion, where the distal portion includes a threaded portion configured to engage bone of the patient and the proximal portion is configured engage the opening in the proximal portion of the implant body. In some cases, the screw engages both the ilium and the sacrum of the patient. In some cases, the screw is a first screw configured to engage the sacrum, and the system further includes a second screw configured to engage the ilium. In some cases, the proximal portion of the screw locks rigidly to the opening in the implant body. In some cases, the implant body includes a porous structure configured for bony ingrowth. In some cases, the implant body has a window configured to contain bone graft. In some cases, the implant body has an hourglass shaped cross-section having a narrow section configured to be on the superior and inferior sides of the implant, and a narrowest point configured to be located at a sacroiliac joint of the patient. In some cases, the screw is a first screw, and the system further includes a second screw configured to engage the ilium and the sacrum, and the second screw is substantially perpendicular to a long axis of the implant body. In some cases, the second screw has a threaded portion configured to engage the implant body. In some cases, a head of the second screw compresses the ilium to the implant body. In some cases, a head of the second screw compresses the sacrum to the implant body. In some cases, the head of the second screw compresses the ilium to the sacrum. In some cases, the screw is a first screw, the system further includes a second screw configured to engage the ilium and the sacrum, and the second screw engages a long axis of the implant body at an acute angle.

In another aspect, embodiments of the present invention encompass systems and methods for performing a sacroiliac fusion procedure on a patient. Exemplary methods can include placing a guide pin in a plane of a sacroiliac joint of the patient, removing sacral and iliac bone of the patient to create a receptacle for an implant body, inserting the implant body in the receptacle, and securing the implant body to the patient with a screw. In some cases, the implant body has a medial side configured to engage a sacrum of a patient, a lateral side configured to engage an ilium of the patient, a distal portion, and a proximal portion having an opening configured to receive the screw. In some cases, the screw has a distal portion and proximal portion, where the distal portion has a threaded portion configured to engage bone of the patient and the proximal portion is configured engage the opening in the proximal portion of the implant body. In some cases, the screw engages both the ilium and the sacrum of the patient. In some cases, the screw is a first screw configured to engage the sacrum, and the system further includes a second screw configured to engage the ilium. In some cases, the proximal portion of the screw locks rigidly to the opening in the implant body. In some cases, the implant body has a porous structure configured for bony ingrowth. In some cases, the implant body has a window configured to contain bone graft. In some cases, the implant body has an hourglass shaped cross-section having a narrow section configured to be on the superior and inferior sides of the implant, and a narrowest point configured to be located at a sacroiliac joint of the patient. In some cases, the screw is a first screw, the system further includes a second screw configured to engage the ilium and the sacrum, and the second screw is substantially perpendicular to a long axis of the implant body. In some cases, the second screw has a threaded portion configured to engage the implant body. In some cases, a head of the second screw compresses the ilium to the implant body. In some cases, a head of the second screw compresses the sacrum to the implant body. In some cases, the head of the second screw compresses the ilium to the sacrum. In some cases, the screw is a first screw, the system further includes a second screw configured to engage the ilium and the sacrum, and the second screw engages a long axis of the implant body at an acute angle. In some embodiments, the step of removing sacral and iliac bone of the patient includes using a box cut device. In some embodiments, the step of inserting the implant body in the receptacle includes using an inserter device. In some embodiments, the step of removing the sacral and iliac bone or inserting the implant body includes using an outrigger device.

Additional aspects of embodiments of the invention will be apparent from the detailed descriptions and claims herein.

### BRIEF DESCRIPTION OF THE DRAWINGS

Novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIGS. 1A to 1N illustrate aspects of various sacroiliac fusion systems and methods, in accordance with some embodiments;

FIGS. 2A to 2G illustrate aspects of various sacroiliac fusion systems and methods, in accordance with some embodiments;

FIGS. 3A and 3B depict aspects of various sacroiliac fusion systems and methods, in accordance with some embodiments;

FIGS. 4A and 4B depict aspects of various sacroiliac fusion systems and methods, in accordance with some embodiments;

FIGS. 5A to 5C illustrate aspects of various sacroiliac fusion systems and methods, in accordance with some embodiment;

FIGS. 6A to 6F illustrate aspects of various interbody configurations, in accordance with some embodiments;

FIGS. 7A to 7E illustrate aspects of various sacroiliac fusion systems and methods, in accordance with some embodiments;

FIGS. 8A to 8C illustrate aspects of various sacroiliac fusion systems and methods, in accordance with some embodiments;

FIGS. 9A to 9W illustrate aspects of various sacroiliac fusion systems and methods, in accordance with some embodiments;

FIGS. 10A to 10I illustrate aspects of various sacroiliac fusion systems and methods, in accordance with some embodiments;

FIGS. 11A to 11G illustrate aspects of various sacroiliac fusion systems and methods, in accordance with some embodiments;

FIGS. 12A and 12B depict aspects of various sacroiliac fusion systems and methods, in accordance with some embodiments;

FIGS. 13A and 13B depict aspects of various sacroiliac fusion systems and methods, in accordance with some embodiments;

FIGS. 14A and 14B depict aspects of various sacroiliac fusion systems and methods, in accordance with some embodiments;

FIGS. 15A and 15B depict aspects of a broach device, according to embodiments of the present invention;

FIGS. 16A and 16B depict aspects of an inserter device, according to embodiments of the present invention; and

FIGS. 17A and 17B depict aspects of an outrigger instrument, according to embodiments of the present invention.

### DETAILED DESCRIPTION

Specific embodiments of the disclosed device, system and method of use will now be described with reference to the drawings. Nothing in this detailed description is intended to imply that any particular component, feature, or step is essential to embodiments of the invention.

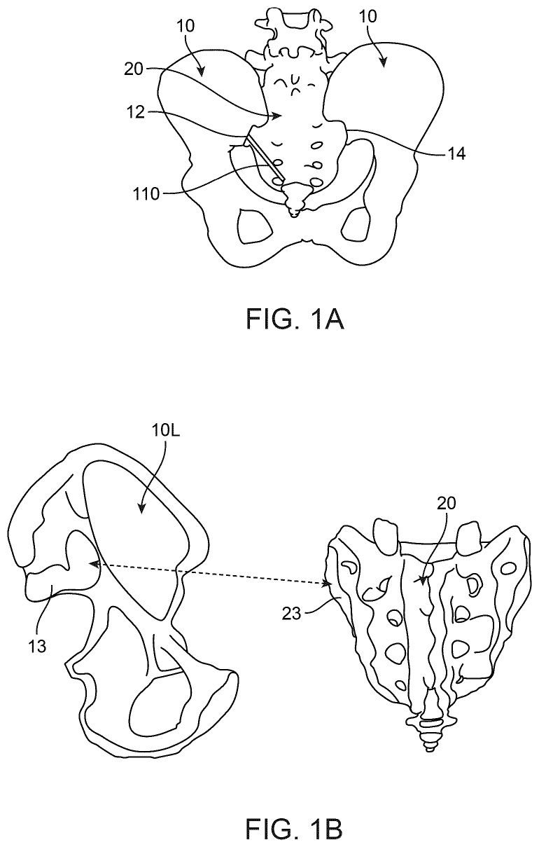

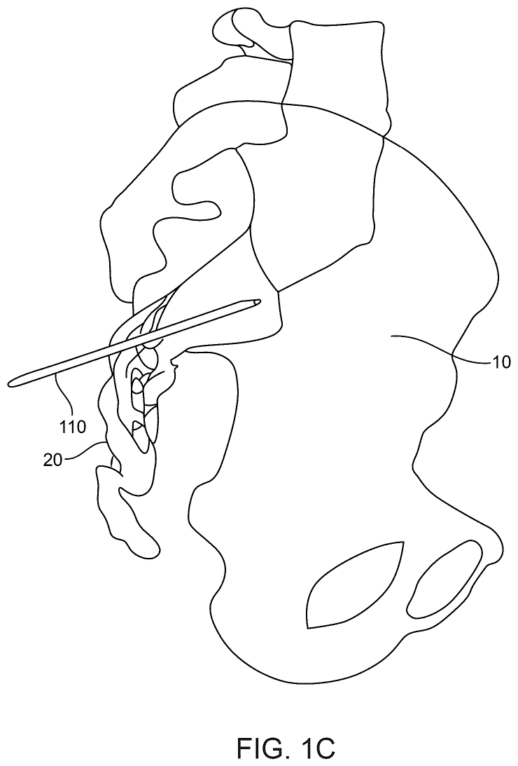

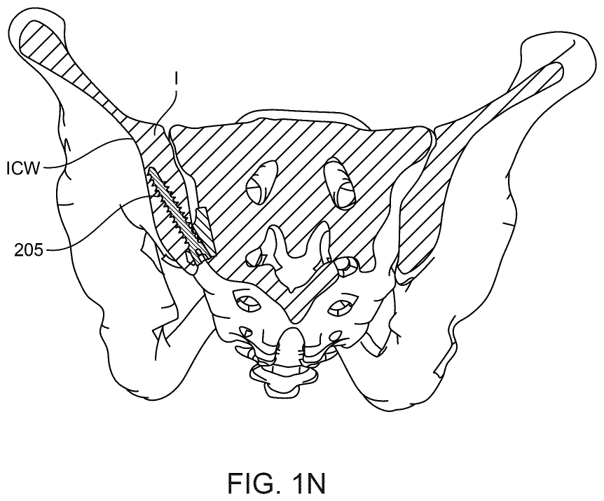

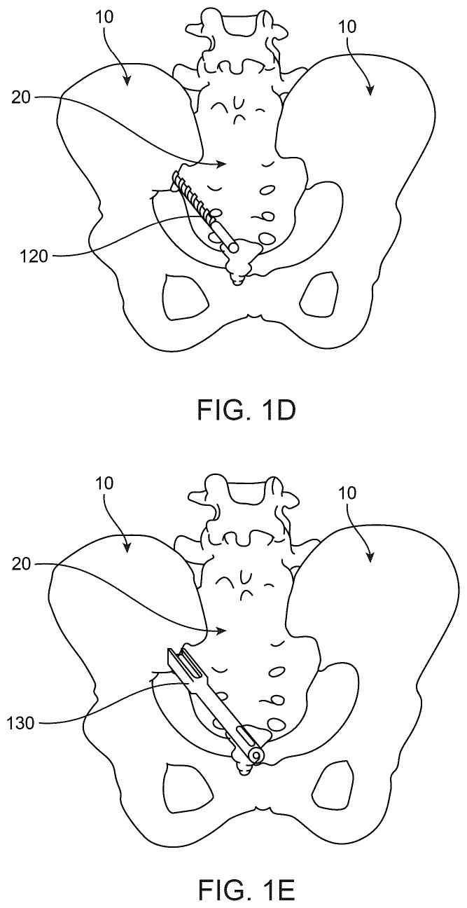

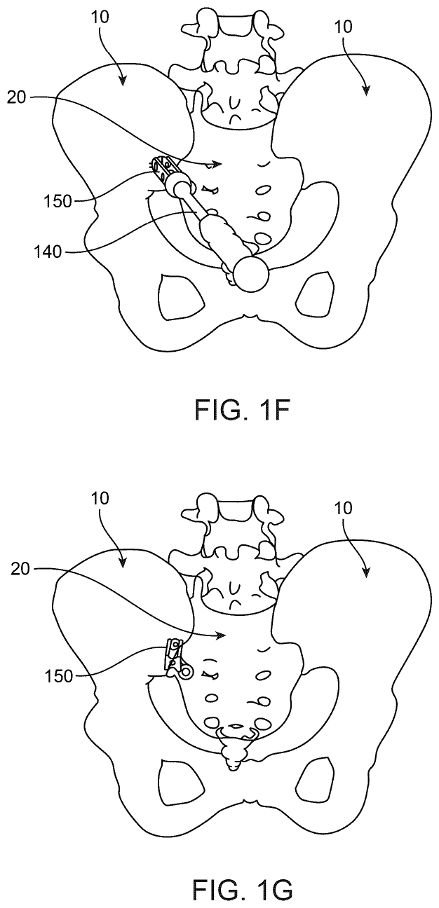

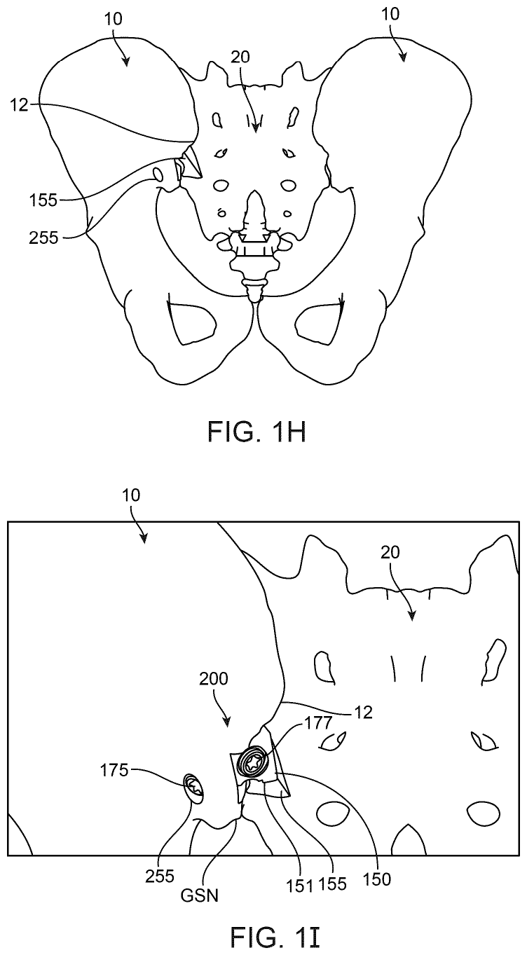

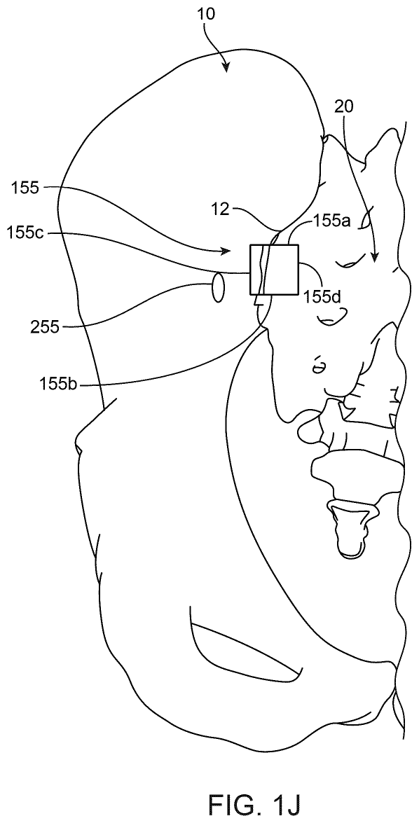

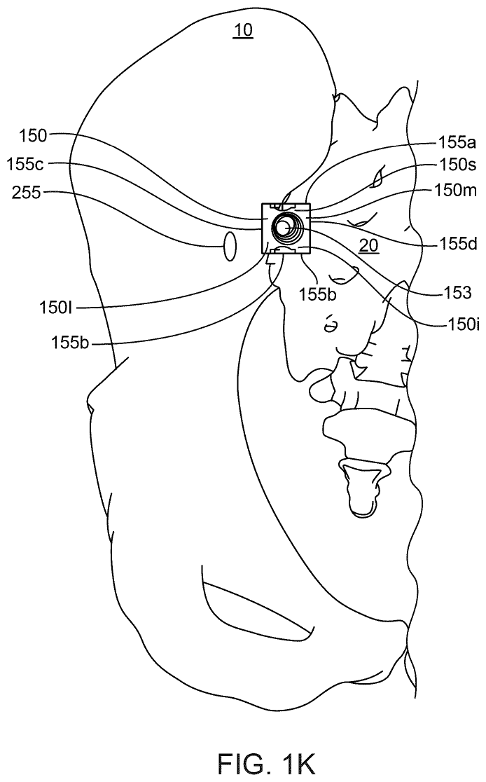

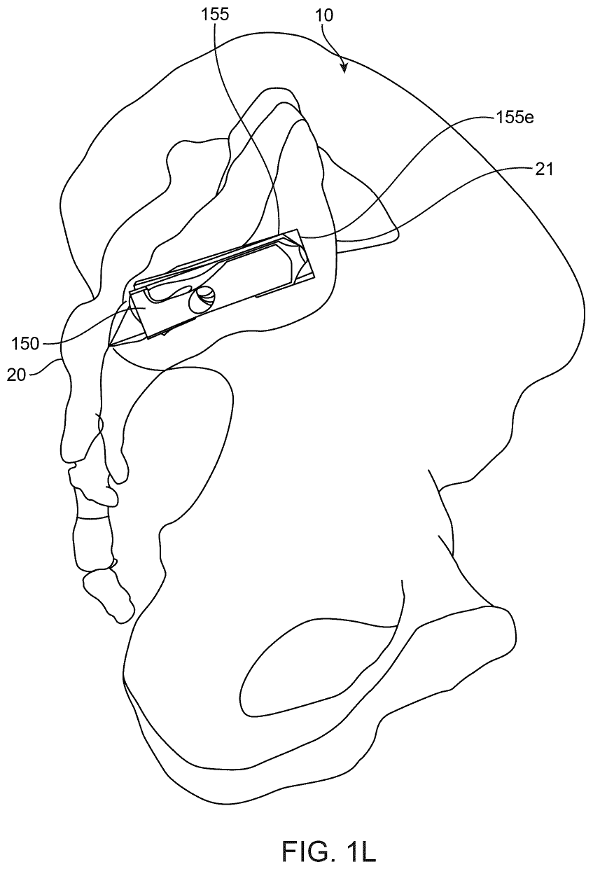

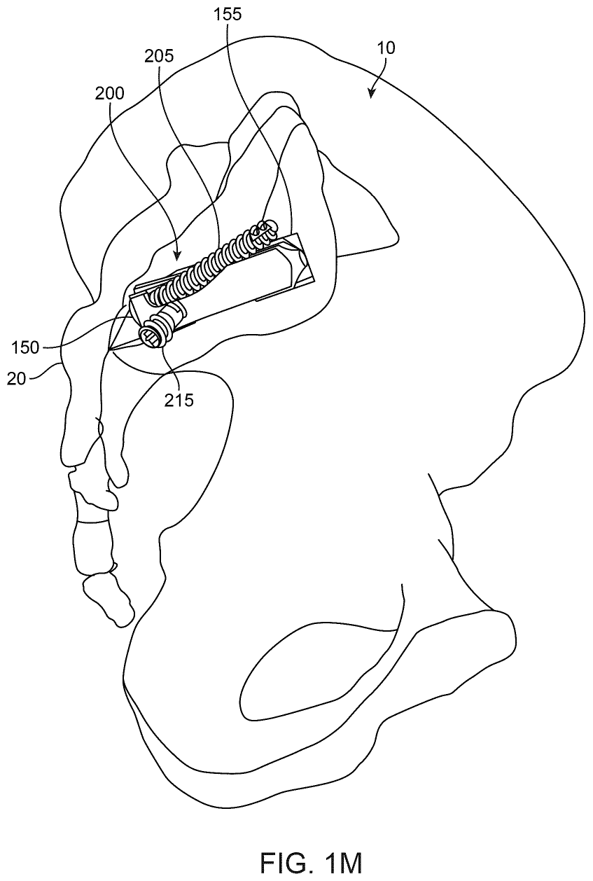

Turning now to the drawings, FIG. 1A provides a posterior view of the pelvis bones of an individual or patient. As shown here, the ilium 10 is coupled with the sacrum 20 via a first sacroiliac joint 12 and a second sacroiliac joint 14. A pin device 110 can be inserted into joint space, for example the first sacroiliac joint space 12 of the patient. FIG. 1B provides an exploded illustration of a medial view of the left wing 10L of the ilium and a posterior view of the sacrum 20. The ilium includes an auricular surface 13 that interfaces with a left auricular surface 23 of the sacrum. A sacroiliac joint can include the interface between the auricular surface 13 of the ilium and the auricular surface 23 of the sacrum, as shown by dashed arrow A.

FIG. 1C provides a side view of the patient ilium 10 and sacrum 20. These bones are depicted as transparent objects, so that the positioning of the pin device 110 within the patient anatomy can be visualized more easily. As shown here, the pin device 110 can be inserted in the sacroiliac joint or joint space along the interface between the auricular surface of the ilium and the auricular surface of the sacrum. In some embodiments, a surgeon or operator can use the pin device 110 as a guide for a ream device.

FIG. 1D provides a posterior view of the patient ilium 10 and sacrum 20. As shown here, a ream or ream device 120 can be provided, positioned, placed, and/or advanced over the pin. In some embodiments, a surgeon or operator can use the ream device 120 as a guide for a box cut device.

FIG. 1E provides a posterior view of the patient ilium 10 and sacrum 20. As shown here, a box cut or box cut device 130 can be provided, positioned, placed, and/or advanced over the ream device. In some embodiments, a surgeon or operator can use the box cut device 130 to create a channel or receptacle, for example to receive or at least partially receive an interbody.

FIG. 1F provides a posterior view of the patient ilium 10 and sacrum 20. As shown here, an impact tool 140 can be used to impact an interbody device 150 into the joint cavity. In some embodiments, an interbody device can also be referred to as an implant body. In some embodiments, an interbody device can be a component of an implant assembly. In some cases, the impact tool 140 can be used to advance or force the interbody device 150 into or at least partially into the sacroiliac joint space or cavity. In some cases, the impact tool 140 can be used to advance or force the interbody device 150 into or at least partially into a channel or receptacle that has been formed in the sacroiliac joint by the box cut device. In some embodiments, another type of impact tool or inserter can be used.

FIG. 1G provides a posterior view of the patient ilium 10 and sacrum 20, depicting the interbody device 150 in a joint (e.g. sacroiliac joint).

FIG. 1H provides a posterior view of the patient ilium 10 and sacrum 20, depicting an interbody channel 155 which has been formed in the patient anatomy (e.g. at the sacroiliac joint 12) and which is configured to at least partially receive, contain, or support an interbody. In some cases, a surgeon or operator can create an interbody channel 155 by using a box cut or box cut device, as discussed elsewhere herein. Interbody channel 155 can be defined by a combination of ilium and sacrum bone tissue surfaces. FIG. 1H also depicts an engagement channel 255 which is configured to at least partially receive, contain, or support a fixation element such as a screw, for example a posterior cross screw. Any of a variety of instruments or tools can be used to create the engagement channel 255. According to some embodiments, a screw or fixation mechanism can enter the ilium, traverse an interbody (e.g. so no joint is left in that space), and then enter the sacrum. In some cases, a channel 255 can enter the ilium, traverse the sacroiliac joint, and then enters the sacrum. In some cases, a channel 255 may not traverse the joint.

FIG. 1I depicts aspects of an implant assembly 200 which has been placed or implanted in the patient anatomy. As shown here, an interbody 150 of the implant assembly 200 is engaged with the interbody channel 155 and a posterior fixation mechanism 175 of the implant assembly 200 is engaged with the engagement channel 255. In this embodiment, implant assembly 200 also includes a plate fixation mechanism 177, which in some cases can include a plate screw. In some instances, one or more of the implant assembly components are configured to lay flush or subflush to the surrounding anatomy of the patient (e.g. surface of ilium 10 and/or sacrum 20). In some instances, an inferior side or surface 151 of the implant body is located at a distance having a value of at least 10 mm from the greater sciatic notch GSN of the patient. In some embodiments, an implant assembly may include an anterior fixation mechanism in addition to or instead of posterior fixation mechanism 175. In such cases, the anterior fixation mechanism may be advanced through an anterior bony engagement channel (not shown) in the ilium, which may for example be located in a position that is anterior to the location where channel 255 is depicted here.

FIG. 1J aspects of an interbody channel 155 and an engagement channel 255, according to embodiments of the present invention. In some embodiments, a box cut tool or other mechanism can be used to form either or both of these channels. Interbody channel 155 can have a superior surface 155a, an inferior surface 155b, a lateral surface 155c, and a medial surface 155d. As shown here, superior surface 155a can include both ilium bone and sacrum bone. Likewise, inferior surface 155b can include both ilium bone and sacrum bone. In some instances, lateral surface 155c includes ilium bone, and medial surface 155d includes sacrum bone.

As shown in FIG. 1K, interbody 150 can have a central cannula 153 with a central longitudinal axis that aligns with a trajectory of the sacroiliac joint, or interface between the auricular surface of the ilium 10 and the auricular surface of the sacrum 20. In the embodiment depicted here, the interbody channel is formed in an asymmetrical fashion relative to the ilium and sacrum, so that the interbody has a larger volume purchase on the sacral side as compared with the iliac side. For example, the majority of each of the superior 155a and inferior 155b surfaces is sacral bone. This can enhance the operational effectiveness of the interbody 150, by utilizing the higher density/quality of bone in the sacrum relative to the ilium. When the interbody 150 is placed in the interbody channel or receptacle, a superior side or portion 150s of the interbody can contact or face toward a superior surface 155a of the channel, an inferior side or portion 150i of the interbody can contact or face toward an inferior surface 155b of the channel, a lateral side or portion 150l of the interbody can contact or face toward a lateral surface 155c of the channel, and a medial side or portion 150m of the interbody can contact or face toward a medial surface 155d of the channel.

FIG. 1L provides a side view of the patient ilium 10 and sacrum 20. These bones are depicted as transparent objects, so that the positioning of the interbody 150 and interbody channel 155 within the patient anatomy can be visualized more easily. As shown here, the interbody channel 155 can have a distal or anterior end 155e. In some embodiments, a distance between the anterior end 155e of the channel 155 and the sacral cortical wall 21 can have a value of at least 10 mm. In some cases, it is possible to maximize the length of an interbody 150 for the patient's anatomy by maintaining a distance between the anterior end 155e of the channel 155 and the sacral cortical wall 21 to a certain minimum distance, such as at least 10 mm. In some embodiments, the length of the interbody 150 generally follows the trajectory of the inferior sacral cortical wall. In some embodiments, the positioned interbody 150 can be flush or subflush on all sides of the interbody. In some embodiments, the superior/medial corner of the interbody can be closest to flush, while the inferior/medial side of the interbody can be the most subflush.

FIG. 1M provides a side view of the patient ilium 10 and sacrum 20. These bones are depicted as transparent objects, so that the positioning of the implant assembly 200 and interbody channel 155 within the patient anatomy can be visualized more easily. As shown here, implant assembly 200 can include an interbody 150 having a plate engagement mechanism that engages a plate fixation mechanism 205 such as a plate screw. Interbody 150 can also include a posterior engagement mechanism that engages a posterior fixation mechanism 215 such as a posterior cross screw. In some embodiments, interbody 150 can include an anterior engagement mechanism (not shown) that engages an anterior fixation mechanism such as an anterior cross screw or a lateral cross screw.

FIG. 1N provides a top or superior cross section view of the patient anatomy, illustrating how the plate fixation mechanism or screw 205 engages the ilium bone I of the patient. In some instances, the length of the plate fixation mechanism 205, which may also be referred to as an iliac screw, can be specifically selected for the patient anatomy, based on a longest screw length that can be inserted in the ilium without breaching the iliac cortical wall ICW. In some cases, bone I can be cancellous bone of the ilium.

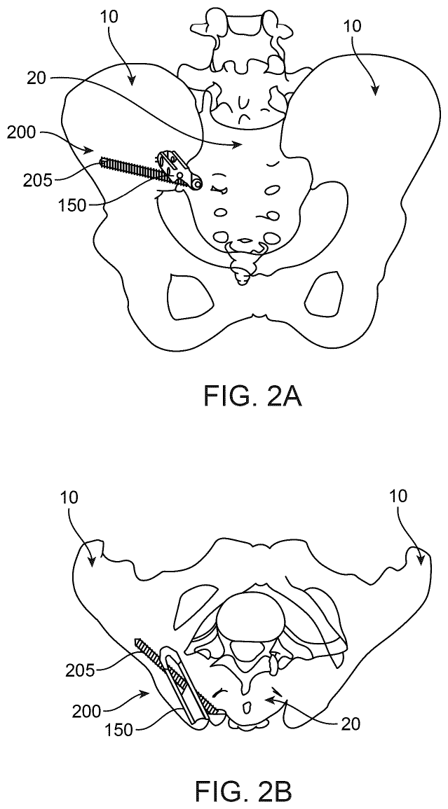

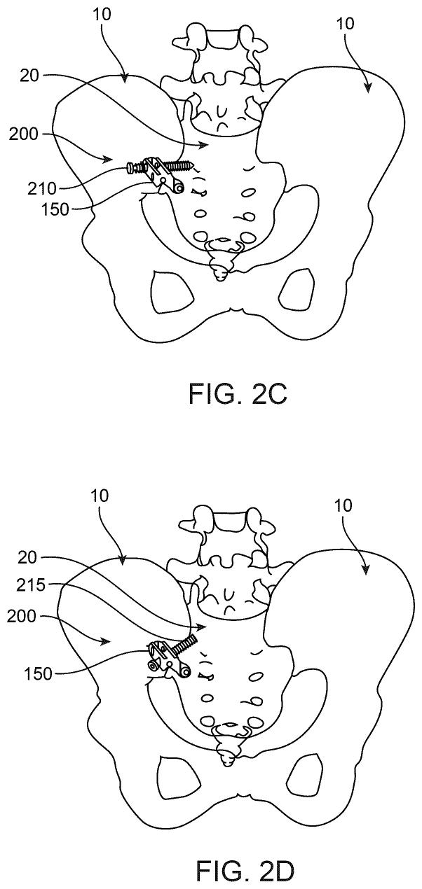

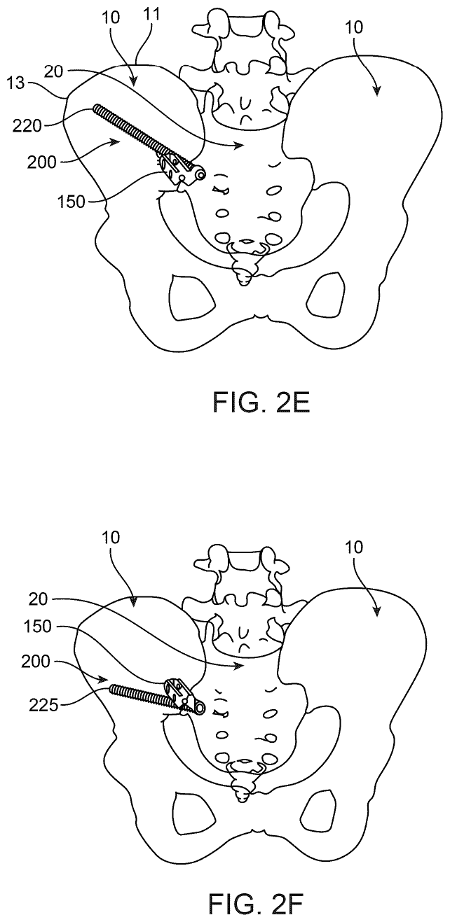

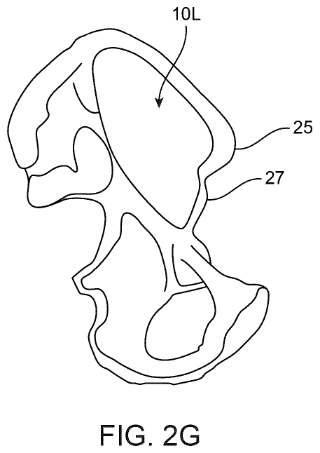

FIG. 2A provides a posterior view of the patient ilium 10 and sacrum 20. These bones are depicted as transparent objects, so that the positioning of the interbody device 150 and screw 205 within the patient anatomy can be visualized more easily. The interbody device 150 is positioned at least partially within the sacroiliac joint, and the screw is positioned at least partially within the ilium 10. In some cases, screw 205 can cross the sacrum 20 partially. In combination, the interbody device 150 and screw 205 can be referred to as components of an implant assembly 200. In some cases, screw 205 can be referred to as a plate fixation mechanism. In some cases, plate fixation mechanism 205 can be advanced along a path in the patient anatomy that is similar to the path that is shown in FIG. 1N.

FIG. 2B provides a top or superior view of the patient ilium 10 and sacrum 20. These bones are depicted as transparent objects, so that the positioning of the interbody device 150 and screw 205 within the patient anatomy can be visualized more easily. The interbody device 150 is positioned at least partially within the sacroiliac joint, and the screw 205 is positioned at least partially within the ilium 10. In some cases, screw 205 can cross the sacrum 20 partially. In combination, the interbody device 150 and screw 205 can be referred to as components of an implant assembly 200.

In some embodiments, screw 205 can be a plate screw. Plate screws can be inserted into a bone of the patient, as applicable or desired.

FIG. 2C provides a posterior view of the patient ilium 10 and sacrum 20. In some embodiments, cross screws can be placed, for example as a component of an implant assembly. FIG. 2C illustrates the placement of a lateral cross screw 210 (e.g. via a secondary incision). The ilium and sacrum are depicted as transparent objects, so that the positioning of the interbody device 150 and screw 210 within the patient anatomy can be visualized more easily. The interbody device 150 is positioned at least partially within the sacroiliac joint, and the screw 210 is positioned at least partially within the sacrum 20. According to some embodiments, a screw or fixation mechanism can enter the ilium, traverse an interbody (e.g. so the interbody has filled the space and there is no joint), and then enter the sacrum. In some cases, the interbody can fill the space, such as the interarticular space or the space between the auricular surface of the ilium and the auricular surface of the sacrum, and there is no joint at that location. In some cases, the screw or fixation mechanism can cross the joint directly if the trajectory is slightly different. In combination, the interbody device 150 and screw 210 can be referred to as components of an implant assembly 200. In some embodiments, screw 210 can be referred to as an anterior fixation mechanism.

FIG. 2D provides a posterior view of the patient ilium 10 and sacrum 20. In some embodiments, cross screws can be placed, for example as a component of an implant assembly. FIG. 2D illustrates the placement of a posterior cross screw 215 (e.g. via an incision migration). The ilium and sacrum are depicted as transparent objects, so that the positioning of the interbody device 150 and screw 215 within the patient anatomy can be visualized more easily. The interbody device 150 is positioned at least partially within the sacroiliac joint, and the screw 215 is positioned at least partially within the sacrum 20. According to some embodiments, a screw or fixation mechanism can enter the ilium, traverse an interbody (e.g. so the interbody has filled the space and there is no joint), and then enter the sacrum. In some cases, the interbody can fill the space, such as the interarticular space or the space between the auricular surface of the ilium and the auricular surface of the sacrum, and there is no joint at that location. In some cases, the screw or fixation mechanism can cross the joint directly if the trajectory is slightly different. In combination, the interbody device 150 and screw 215 can be referred to as components of an implant assembly 200. In some embodiments, screw 215 can be referred to as a posterior fixation mechanism.

FIG. 2E provides a posterior view of the patient ilium 10 and sacrum 20. These bones are depicted as transparent objects, so that the positioning of the interbody device 150 and screw 220 within the patient anatomy can be visualized more easily. In some cases, plate screws can be placed according to different trajectory variations. In some embodiments, such placement may involve a single plate screw. FIG. 2E depicts an exemplary option, showing a superior sacral to iliac screw 220, which may also be referred to as a plate screw. That is, the plate screw can be placed superior to the interbody. Such a trajectory can aim for the superior corner of the iliac crest 11 or the superior iliac crest. In some embodiments, the screw 220 can cross the sacrum and the sacroiliac joint and not cross through the interbody 150 outside of the plate threads.

FIG. 2F provides a posterior view of the patient ilium 10 and sacrum 20. These bones are depicted as transparent objects, so that the positioning of the interbody device 150 and screw 225 within the patient anatomy can be visualized more easily. FIG. 2F depicts an exemplary option, showing an inferior sacral to iliac screw 225. That is, the plate screw is placed inferior to the interbody. Such a trajectory can aim between the anterior inferior iliac spine (AIIS) 27 and the anterior superior iliac spine (ASIS) 25, as shown in the medial view of the left wing 10L of the ilium provided in FIG. 2G. In some embodiments, the screw 225 can cross the sacrum and the sacroiliac joint and not cross through the interbody 150 outside of the plate threads.

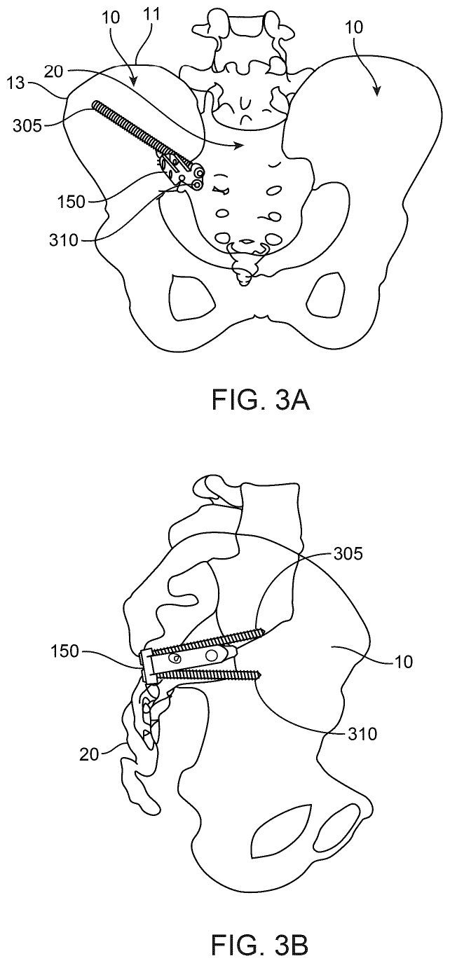

FIG. 3A provides a posterior view of the patient ilium 10 and sacrum 20. FIG. 3B provides a side view of the patient ilium 10 and sacrum 20. These bones are depicted as transparent objects, so that the positioning of the screws 305, 310 and interbody device 150 within the patient anatomy can be visualized more easily. In some embodiments, plate screw placement may involve two plate screws. FIGS. 3A and 3B depict an exemplary option, showing a sacral to iliac screw 305 (e.g. superior sacral to iliac screw) and a sacral screw 310 (e.g. inferior sacral screw). Relatedly, a plate screw (e.g. superior sacral to iliac screw 305) can be placed superior to the interbody 150. The trajectory can aim for the superior corner 13 of the iliac crest 11. In some embodiments, fixation mechanism 310 is a sacral screw, and does not go into the ilium or joint at all, but rather goes directly from the interbody plate to the sacrum. In some embodiments, fixation mechanism 305 is a screw and goes from the interbody plate, across the joint, and into the sacrum.

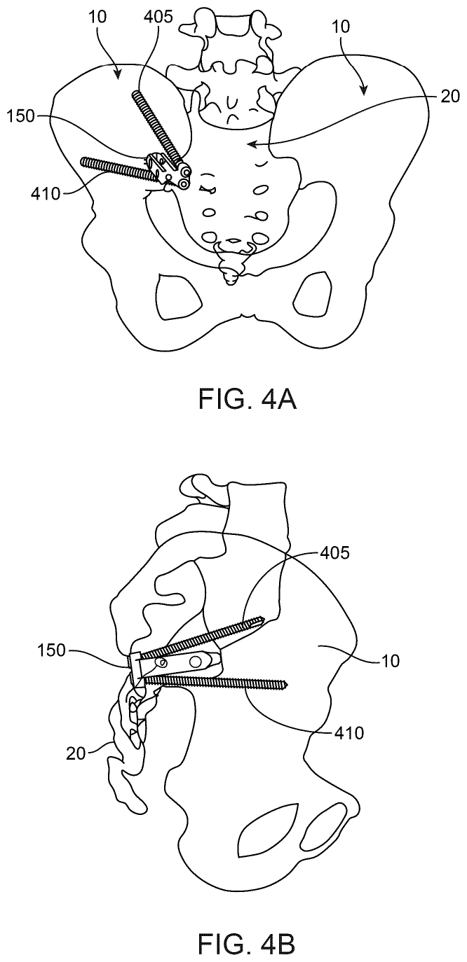

FIG. 4A provides a posterior view of the patient ilium 10 and sacrum 20. FIG. 4B provides a side view of the patient ilium 10 and sacrum 20. These bones are depicted as transparent objects, so that the positioning of the screws 405, 410 and interbody device 150 within the patient anatomy can be visualized more easily. FIGS. 4A and 4B depict an exemplary option, showing a sacral screw 405 (e.g. superior sacral screw) and a sacral to iliac screw 410 (e.g. inferior sacral to iliac screw). Relatedly, a plate screw can be placed inferior to the interbody. The trajectory can aim between the anterior superior iliac spine (ASIS) and the anterior inferior iliac spine (AIIS). In some cases, the sacral to iliac screw 410 can be referred to as a SWAI screw. In some embodiments, fixation mechanism 410 is a sacral screw, and does not go into the ilium or joint at all, but rather goes directly from the interbody plate to the sacrum. In some embodiments, fixation mechanism 405 is a screw and goes from the interbody plate, across the joint, and into the sacrum.

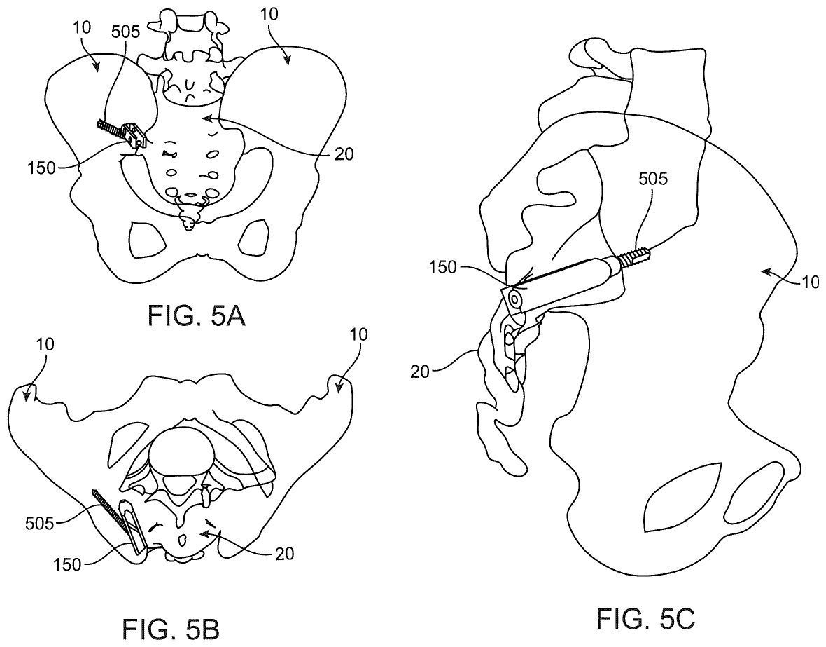

FIG. 5A provides a posterior view of the patient ilium 10 and sacrum 20, FIG. 5B provides a superior or top view of the patient ilium 10 and sacrum 20, and FIG. 5C provides a lateral or side view of the patient ilium 10 and sacrum. These bones are depicted as transparent objects, so that the positioning of the screw 505 and interbody device 150 within the patient anatomy can be visualized more easily. In another plate screw variation, embodiments may involve placing a sacral to iliac screw 505 through an interbody 150, as depicted in FIGS. 5A, 5B, and 5C. In such cases, the trajectory may aim for the anterior superior iliac spine (ASIS). For these embodiments, a plate may be absent. In some embodiments, screw 505 may enter the ilium directly, without passing through the sacroiliac joint or sacrum. In some embodiments, screw 505 may pass through the interbody 150 and not through the sacroiliac joint or sacrum.

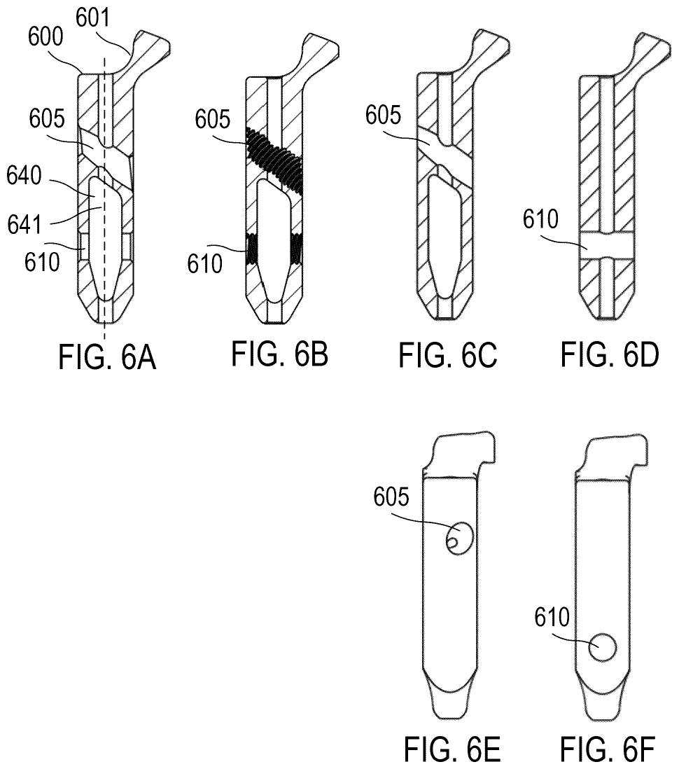

FIGS. 6A to 6F depict aspects of various interbody embodiments. In some cases, an interbody 600 can be a cross screw interbody embodiment. As shown here, an interbody 600 may include a recess, channel, or aperture 605 configured to receive a posterior cross screw. For example, interbody 600 may include recess, channel, or aperture 605 that is configured to receive a screw such as posterior cross screw 215 depicted in FIG. 2D. In some cases, recess, channel, or aperture 605 can be referred to as a posterior engagement mechanism, and posterior engagement mechanism 605 can be configured to engage a posterior fixation mechanism such as a posterior cross screw. In some cases, recess, channel, or aperture 610 can be referred to as an anterior or lateral engagement mechanism, and anterior or lateral engagement mechanism 610 can be configured to engage an anterior or lateral fixation mechanism such as an anterior or lateral cross screw. In some cases, interbody 600 can include a plate engagement mechanism 601, and plate engagement mechanism 601 or plate can be configured to engage a plate fixation mechanism, such as a plate screw.

In some cases, interbody 600 can include an interior section or opening 640 that is configured to receive a graft material. In some cases, opening 640 can be referred to as a central cannula. In some cases, central cannula 640 has with a central longitudinal axis 641 that, when the interbody is implanted in the patient, aligns with a trajectory of the sacroiliac joint, or an interface between the auricular surface of the ilium and the auricular surface of the sacrum. As shown in FIG. 6B, the posterior engagement mechanism 605 and anterior or lateral engagement mechanism 610 can have threaded surfaces. As shown in FIGS. 6C and 6E, an interbody can have a posterior engagement mechanism 605 and no anterior or lateral engagement mechanism. As shown in FIGS. 6D and 6F, an interbody can have an anterior or lateral engagement mechanism 610 and no posterior engagement mechanism.

In some case, an interbody 600 may include a recess, channel, or aperture 610 configured to receive a lateral cross screw. As shown in FIG. 6B, a recess, channel, or aperture can be threaded (e.g. machine threaded). In some cases, a cross screw can be an optional step. In some cases, only one trajectory may be used. In some cases, a posterior option can be completed via an incision migration. In some cases, a lateral option may include a secondary incision. In some cases, a cross screw may thread into the interbody. In some cases, a cross screw may not thread into the interbody.

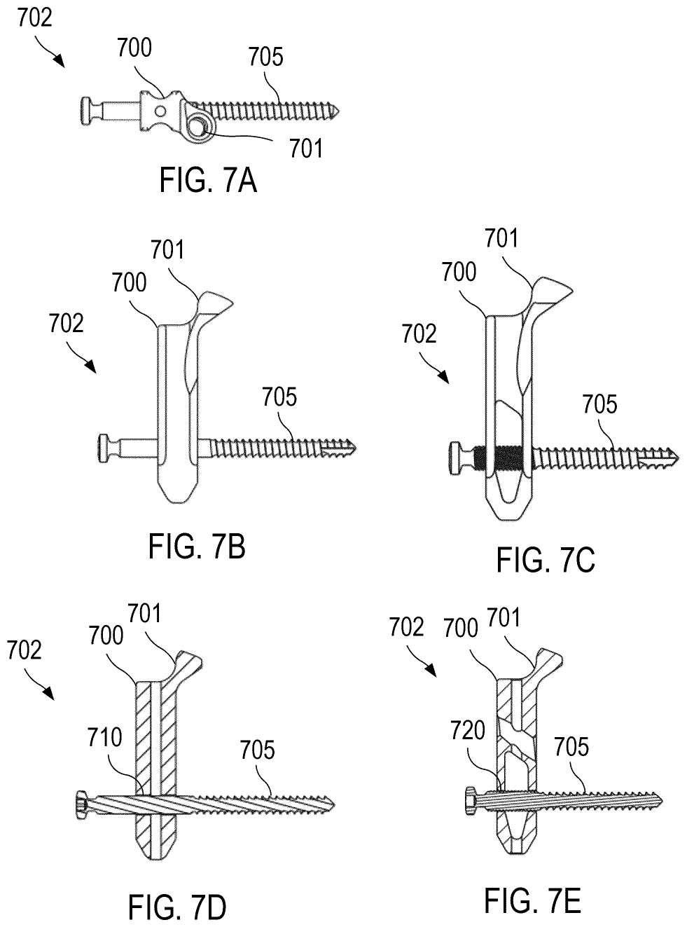

FIGS. 7A to 7E depict aspects of various embodiments involving an interbody 700 and a cross screw 705 (e.g. in an assembled configuration). As shown in FIG. 7D, in some cases the assembly can include a clearance fit 710. As shown in FIG. 7E, in some cases the assembly can include a threaded engagement 720 between the interbody and the cross screw. In some cases, the interbody 700 and screw 705 can be components of an implant assembly 702. In some cases, interbody 700 can include a plate engagement mechanism 701 or plate.

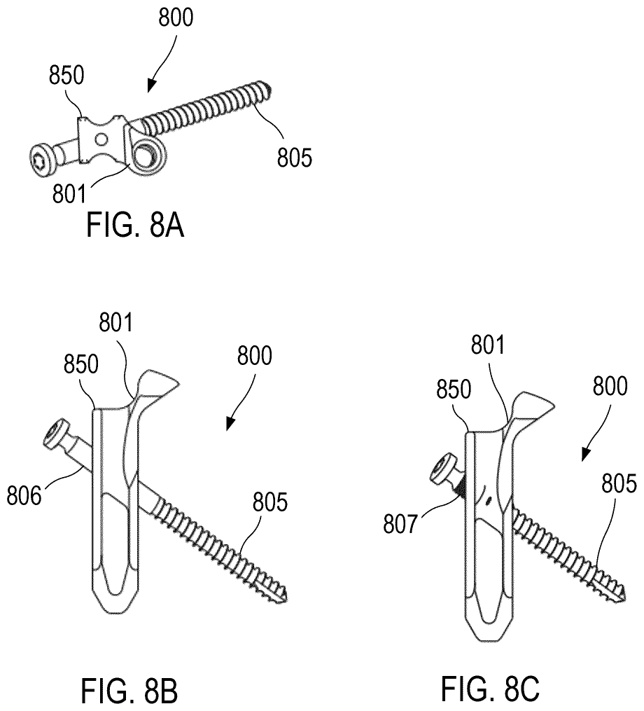

FIGS. 8A to 8C depict various embodiments for an interbody and cross screw assembly 800. In some cases, cross screw assembly 800 can be referred to as an implant assembly. In some cases, implant assembly 800 includes an interbody 850 and a posterior fixation mechanism 805 such as a posterior cross screw. As shown in FIG. 8B, a posterior fixation mechanism 805 can have a proximal or upper section 806 that is not threaded. As shown in FIG. 8C, a posterior fixation mechanism can have a proximal or upper section 807 that is threaded, and such threading can provide additional rigidity to the assembly. In some cases, interbody 850 can include a plate engagement mechanism 801 or plate.

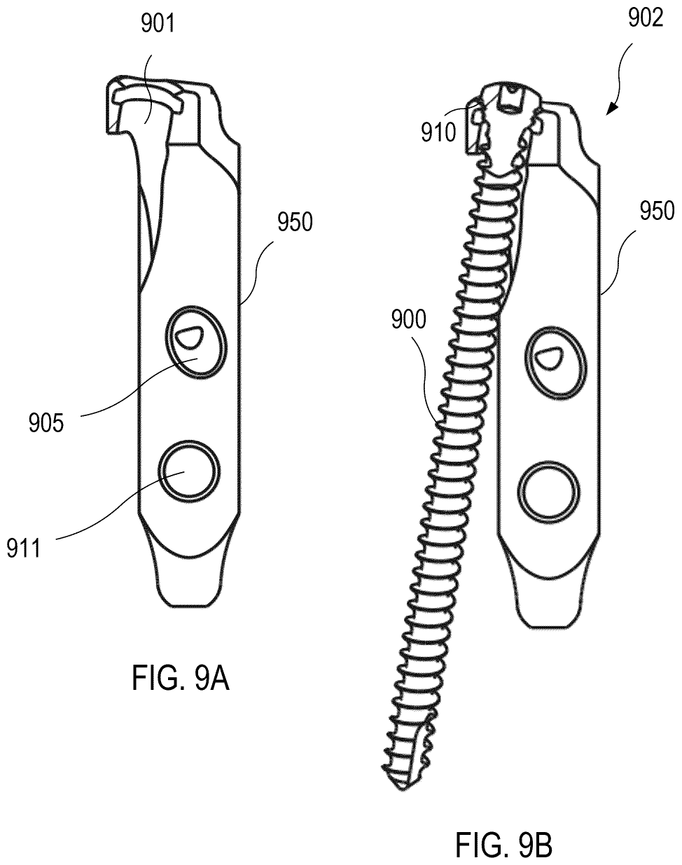

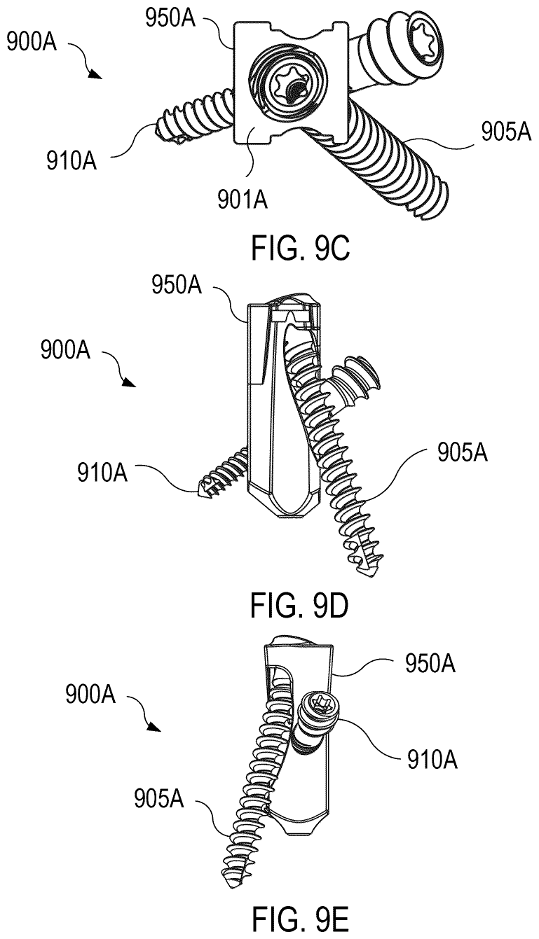

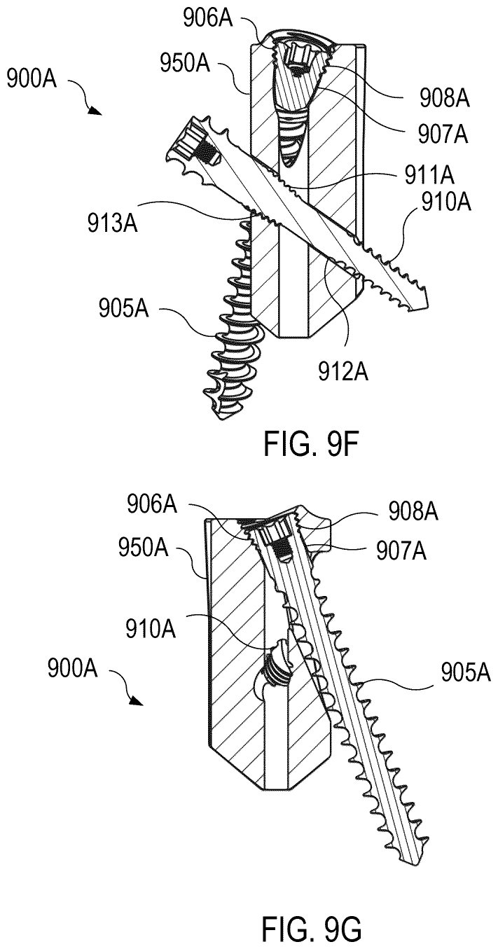

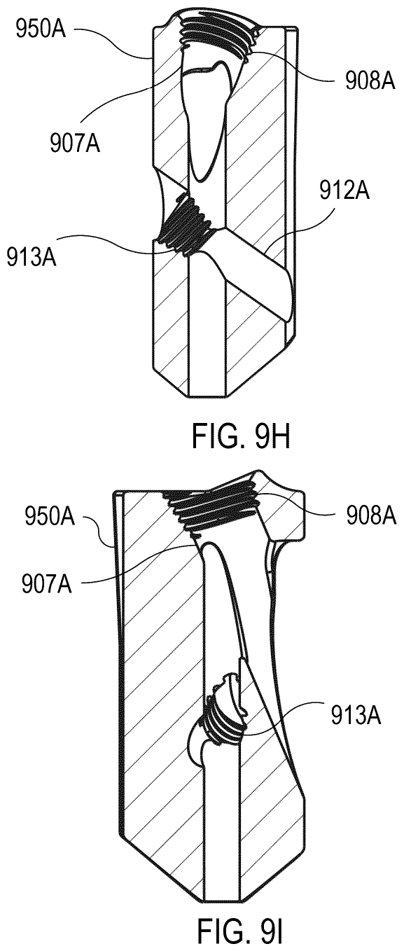

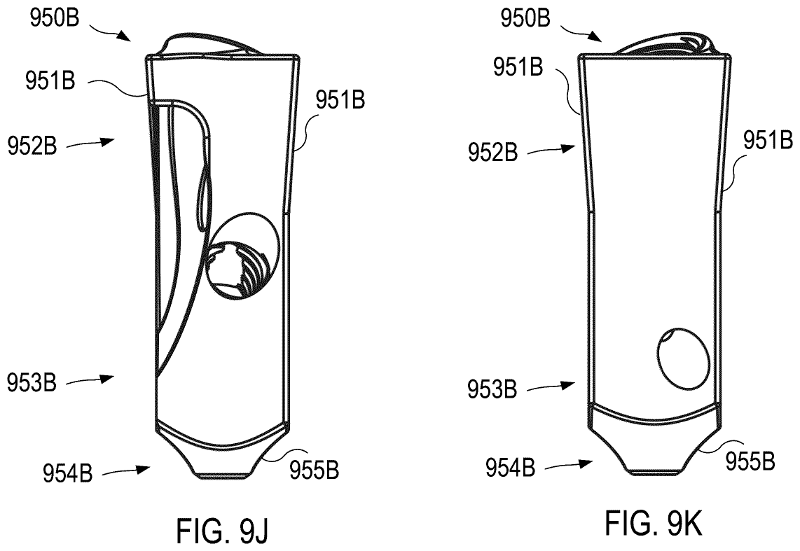

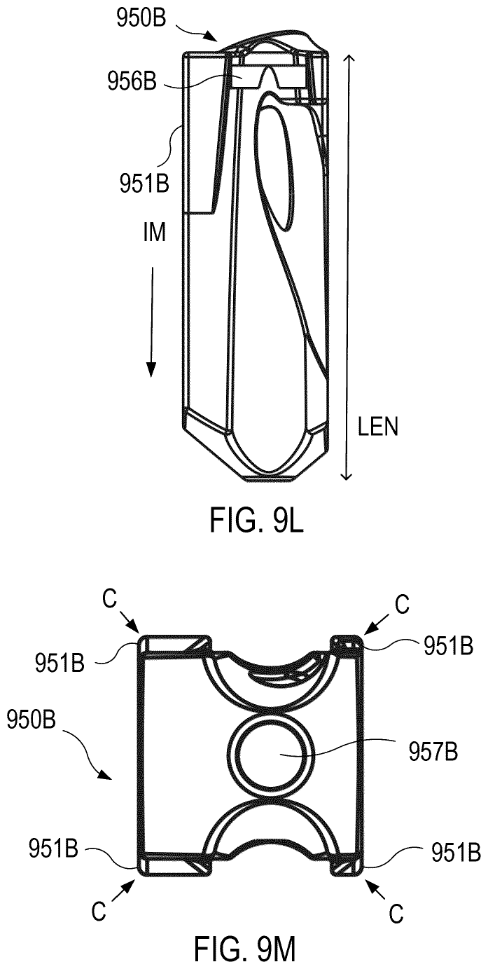

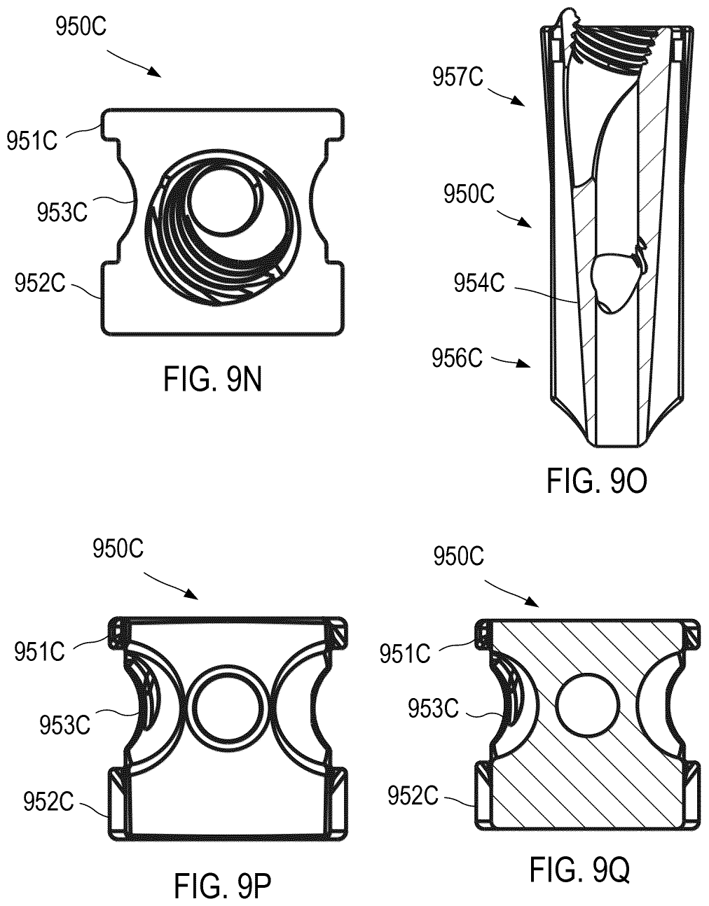

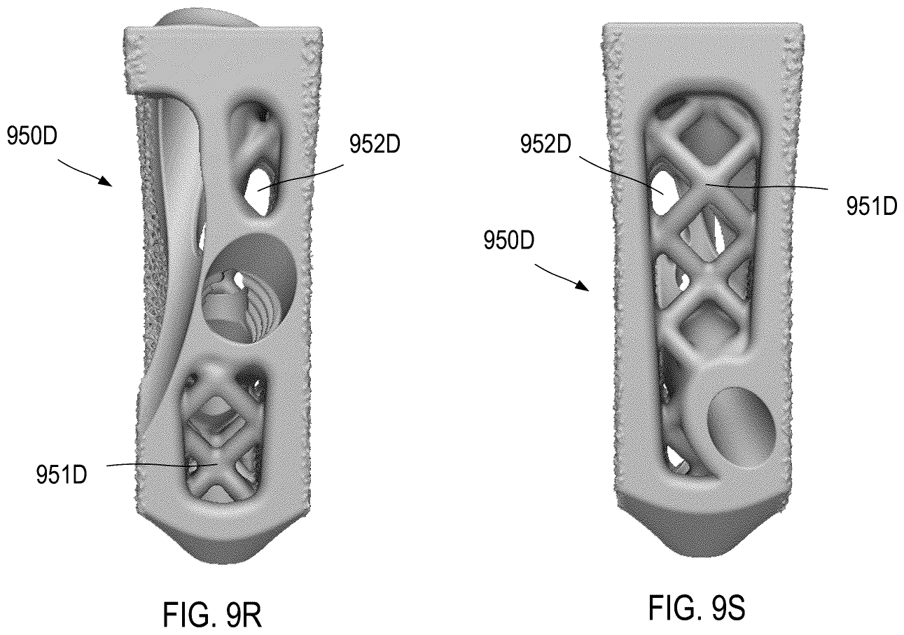

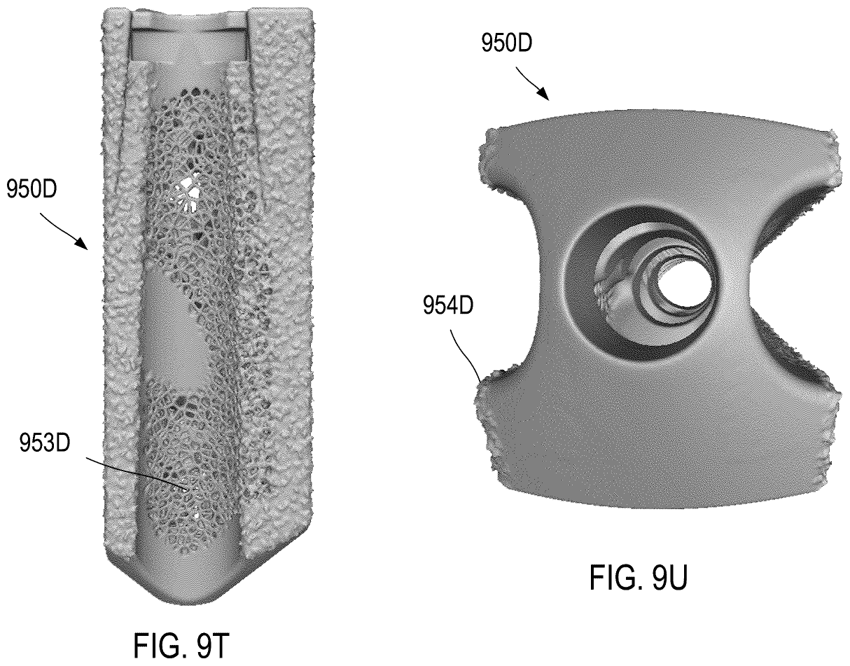

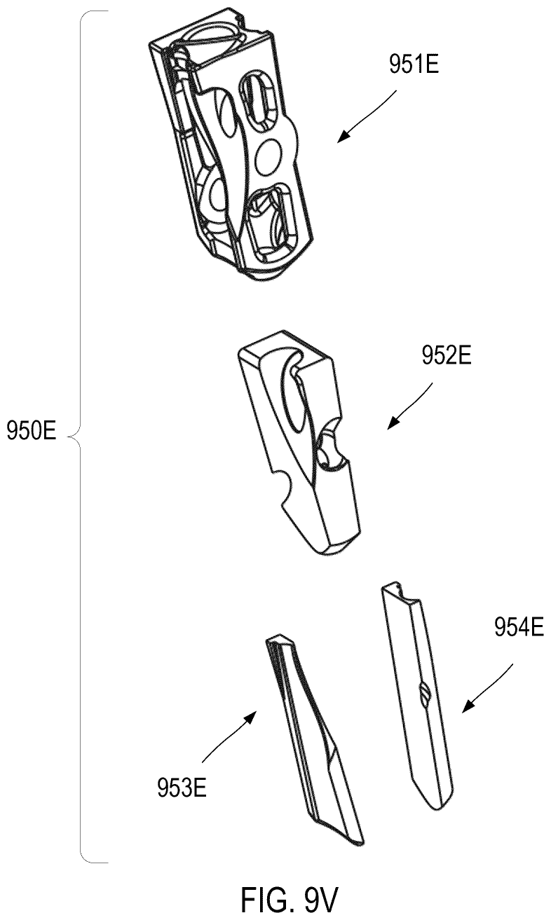

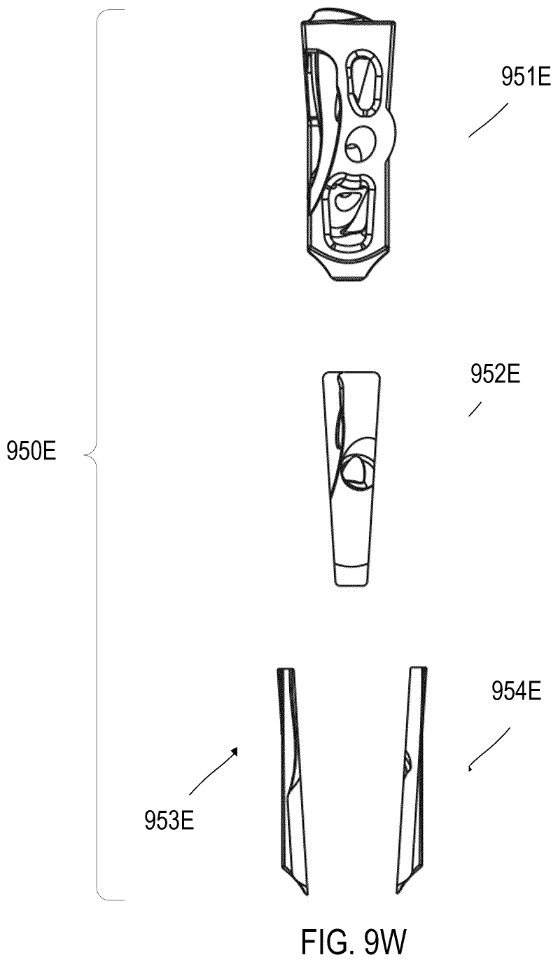

As illustrated in FIGS. 9A and 9B, a plate screw 900 can have a variable angle mechanism 910, which is configured to allow the screw to vary by 5 to 15 degrees. Such variable angle mechanisms can allow for a desired or optimized trajectory via imaging. In some cases, an implant assembly 902 can include an interbody 950 and a plate screw 900. As depicted in FIG. 9A, an interbody can include a plate engagement mechanism 901, a posterior engagement mechanism 905, and an anterior or lateral engagement mechanism 911.

FIGS. 9C to 9E depict aspects of an implant assembly 900A according to embodiments of the present invention. In some cases, the terms “implant assembly” and assembled implant” may be used interchangeably. As shown here, implant assembly 900A can include an interbody 950A, a plate fixation mechanism 905A such as an iliac screw, and a posterior fixation mechanism 910A such as a posterior screw. In some cases, posterior fixation mechanism 910A can be referred to as a transfix screw. Interbody 950A can include a plate engagement mechanism that has a plate 901A. As shown here, plate 901A can have the shape or profile of an I-Beam. In some cases, plate 901A can be referred to as a posterior plate.

FIGS. 9F and 9G depict aspects of an implant assembly 900A according to embodiments of the present invention. As shown here, implant assembly 900A can include an interbody 950A, a plate fixation mechanism 905A such as an iliac screw, and a posterior fixation mechanism 910A such as a posterior screw. In some embodiments, fixation mechanisms can have fixation features that engage with engagement features of engagement mechanisms. For example, plate fixation mechanism 905A can have fixation features 906A such as screw threads that engage with or lock into the interbody 950A. As shown here, interbody 950A can have a plate engagement mechanism 907A such as an aperture which has a plate engagement feature 908A such as a threaded portion. In this sense, fixation feature 906A or threads of plate fixation mechanism 905A can couple with engagement feature 908A of plate engagement mechanism 907A. Likewise, posterior fixation mechanism 910A can have fixation features 911A such as screw threads that engage with or lock into the interbody 950A. As shown here, interbody 950A can have a posterior engagement mechanism 912A such as an aperture which has a posterior engagement feature 913A such as a threaded portion. In this sense, fixation feature 911A or threads of posterior fixation mechanism 910A can couple with engagement feature 913A of posterior engagement mechanism 912A. Aspects of such engagement mechanisms and features are also depicted in FIGS. 9H and 91. In some cases, plate engagement mechanism 907A and/or posterior engagement mechanism 912A can have a fixed trajectory. In some cases, plate engagement feature 908A and/or posterior engagement feature 913A can be referred to as interarticular locking threads.

FIGS. 9J to 9M depict aspects of an interbody 950B according to embodiments of the present invention. Interbody 950B can also be referred to as an interarticular implant. As shown here, an interbody can have one or more compression tapers 951B. The tapers can be positioned at a posterior section 952B of the interbody. Hence, the width and/or depth of the interbody at the posterior section 952B can be greater than the width and/or depth of the interbody at the anterior section 953B. The length LEN of an interbody 950B can be configured so as to accommodate a specific patient anatomy. Hence, different interbody embodiments can be manufactured or produced in various lengths. As shown here, an anterior tip 954B of the interbody can have a tapered section or shape 955B which can aid with insertion into a channel which has been prepared in the patient tissue or bone. In some embodiments, a taper can extend out laterally from the interbody, with increasing width toward the posterior side of the interbody. A taper can allow for bone compression as the interbody is impacted into its prepared channel (e.g. in the direction indicated by arrow IM in FIG. 9L). In some embodiments, a taper can be present on the sacral and iliac sides in the superior/inferior direction. As shown here, an interbody can have four tapers 951B, one on each corner. As illustrated in FIG. 9M, as an interbody 950B is advanced into a channel in the patient anatomy (e.g. at sacroiliac joint) compression forces C from the surrounding tissue or bone act upon the four corners of the interbody. As illustrated in FIG. 9L, an interbody 950B can have an alignment mechanism 956B that enables precise alignment and fixturing with an instrumentation device such as a inserter and removal tool. As illustrated in FIG. 9M, an interbody 950B can include a central cannula or through hole 957B for graft deployment. In some cases, central cannula 957B can provide an interior section or opening that is configured to receive a graft material.

In some embodiments, an interbody can have an I-Beam configuration. FIG. 9N, illustrates a posterior side of an interbody 950C having an I-Beam configuration. In this sense, a posterior plate of the interbody can have an I-Beam shape or outline. The I-Beam configuration provides a first wide section 951C, a second wide section 952C, and a narrow section 953C disposed between sections 951C and 952C. As shown in FIG. 9O, an interbody 950C can have an I-Beam taper 954C. FIG. 9P provides an inferior view of the anterior section of the interbody 950C. FIG. 9Q provides a cross section view of the interbody 950C. The I-Beam taper can be configured to aid with fixation. In some cases, the I-Beam can taper so that the I-Beam is more pronounced on the distal/inferior side (e.g. anterior section 956C) of the interbody 950C as compared with the proximal/superior side (e.g. posterior section 957C) of the interbody. For example, the narrow section 953C shown in FIG. 9P is smaller than the narrow section 953C shown in FIG. 9N.

In some embodiments, an interbody may have one or more porous features. As shown in FIGS. 9R and 9S, an interbody 950D can have a solid frame 951D that provides structural stability and strength and one or more openings 952D configured to receive graft material. As shown in FIG. 9T, interbody 950D can have a porous layer 953D configured to receive or facilitate bony ingrowth therein. As shown in FIG. 9U, interbody 950D can have a surface texture 954D that is configured to receive or enhance ingrowth therein.

FIG. 9V provides an exploded assembly view of an interbody 950E according to embodiments of the present invention. As shown here, interbody 950E can include a solid frame section 951E, an internal graft section 952E, a first ingrowth section 953E having porous and/or surface texture features and a second ingrowth section 954E having porous and/or surface texture features. FIG. 9W provides another exploded assembly view of interbody 950E according to embodiments of the present invention. As shown here, interbody 950E can include a solid frame section 951E, an internal graft section 952E, a first ingrowth section 953E having porous and/or surface texture features and a second ingrowth section 954E having porous and/or surface texture features. In some embodiments, an interbody such as interbody 950E depicted in FIG. 9V or 9W can be a monolithic construct, and for example can be 3D printed as a single component. In some embodiments, a titanium printer can sinter powder to create such a single construct. The discrete sections shown here can be compiled or assembled in a computer program to create a single component that is then manufactured. According to some embodiments, the different components illustrated here depict different sections of the implant. According to some embodiments, a computer aided or assembled complete final version of such components or sections can be seen at, for example, FIGS. 9R to 9T.

In some cases, the terms “interbody”, “interbody device”, “interarticular implant”, and “implant body” may be used interchangeably. In some cases, an implant body has an hourglass shaped cross-section. An implant body can include a narrow section configured to be on the superior and inferior sides of the implant, and a narrowest point configured to be located at a sacroiliac joint of the patient. In some cases, an implant body can have a cross-section shape that is square, rectangular, round, semi-round, oval, or any desired shape.

With reference to FIGS. 6A to 9V, for example, it can be seen that a sacroiliac fusion system can include an implant body and one or more screws or fixation mechanisms. In some cases, a sacroiliac fusion system includes an implant body and one screw. In some cases, a sacroiliac fusion system includes an implant body and multiple screws. The screw or screws can be configured to engage a long axis of the implant body at any desired angle. In some cases, a screw is configured to engage a long axis of the implant body at a perpendicular angle. In some cases, a screw is configured to engage a long axis of the implant body at an acute angle. In some cases, a screw is configured to engage a long axis of the implant body at an obtuse angle.

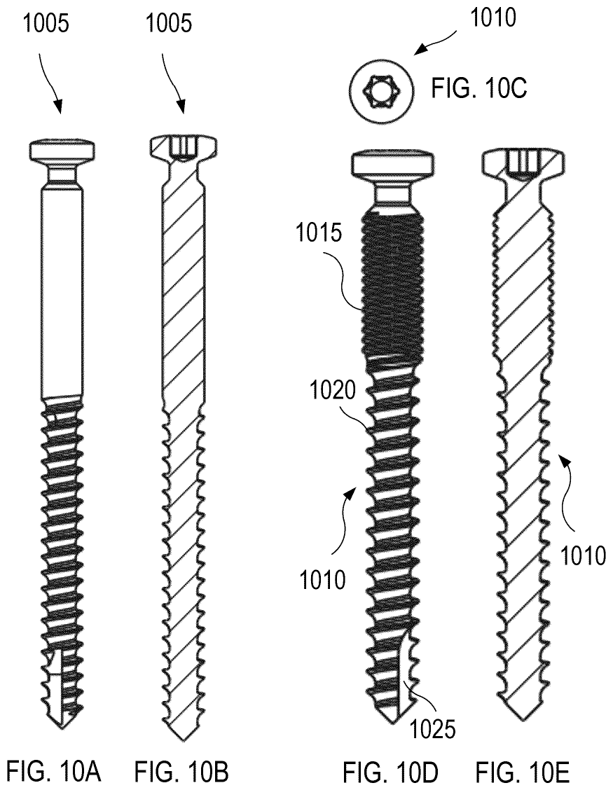

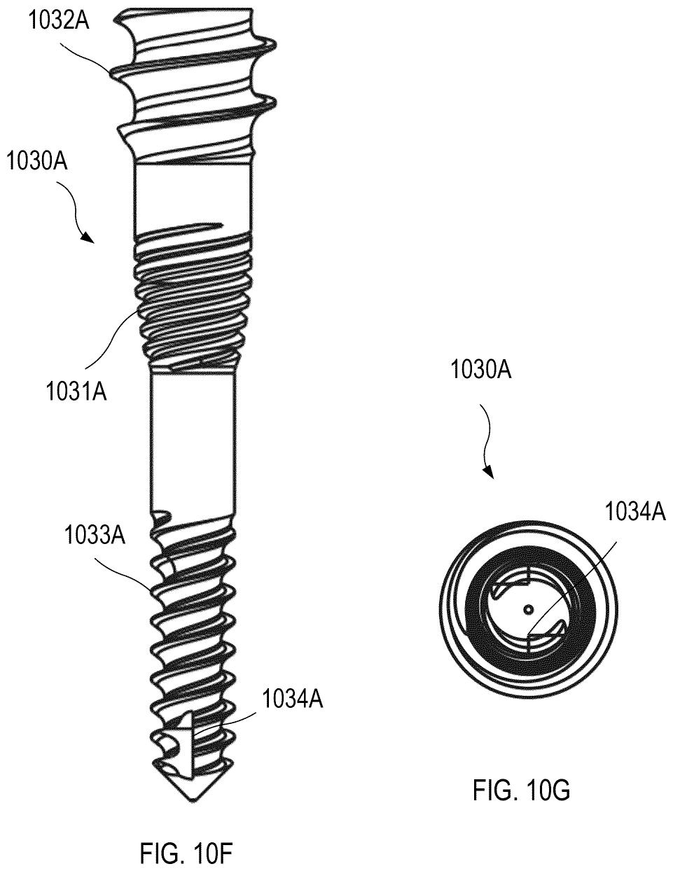

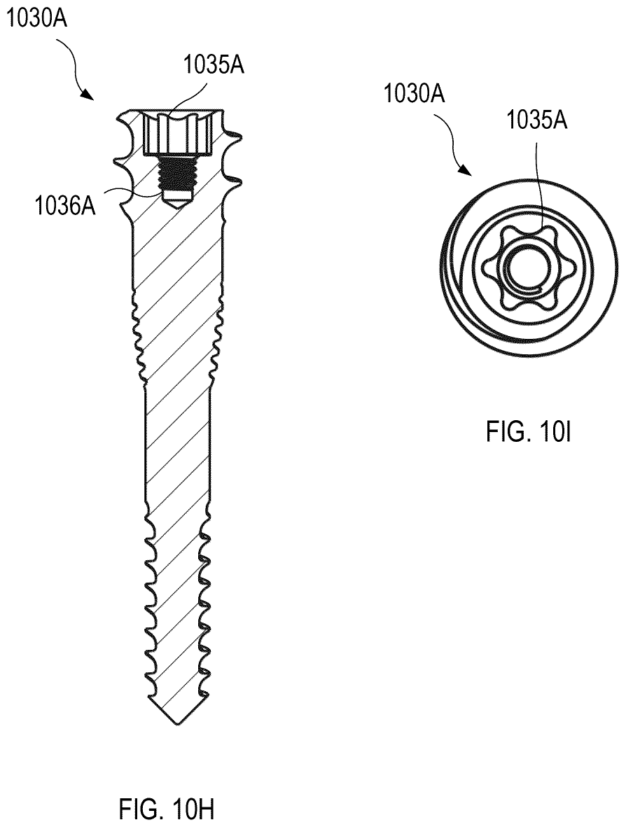

FIGS. 10A to 10E depict aspects of various cross screw configurations, according to embodiments of the present invention. As shown in FIGS. 10A and 10B, a cross screw 1005 can be configured to engage an interbody without threading. As shown in FIGS. 10C to 10E, a cross screw 1010 can be configured to engage an interbody via a threaded mechanism 1015 (e.g. machine threads). In some cases, a cross screw can include bone threads 1020. In some cases, a cross screw can include a self-tapping mechanism 1025. In some cases, cross screw 1005 or 1010 can be referred to as a posterior fixation mechanism. FIGS. 10F and 10G depict aspects of a posterior fixation mechanism 1030A, according to embodiments of the present invention. In some cases, posterior fixation mechanism 1030A can be referred to as a transfix screw. As shown here, a posterior fixation mechanism 1030A can include locking threads 1031A configured for fixation with an interarticular implant or interbody. Posterior fixation mechanism 1030A can also include bone screw threads 1032A (e.g. posterior bone screw threads) for fixation into patient tissue or bone, for example patient ilium bone. Posterior fixation mechanism 1030A can also include bone screw threads 1033A (e.g. anterior bone screw threads) for fixation into patient tissue or bone, for example patient sacrum bone. Posterior fixation mechanism 1030A may also include a self-tapping feature 1034A. As depicted in FIGS. 10H and 10I, a posterior fixation mechanism 1030A can include a driving feature 1035A, such as a T-25 hexalobular driving feature. In some cases, plate fixation mechanism 1030A can include a retaining feature 1036A for a driver.

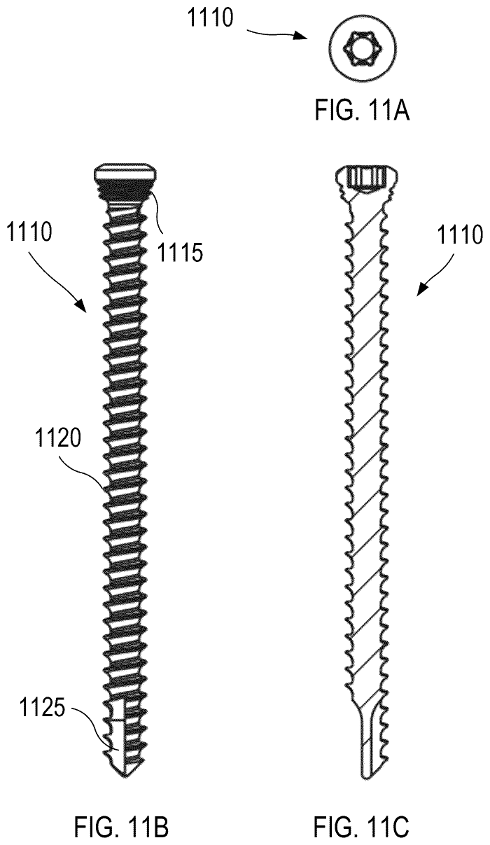

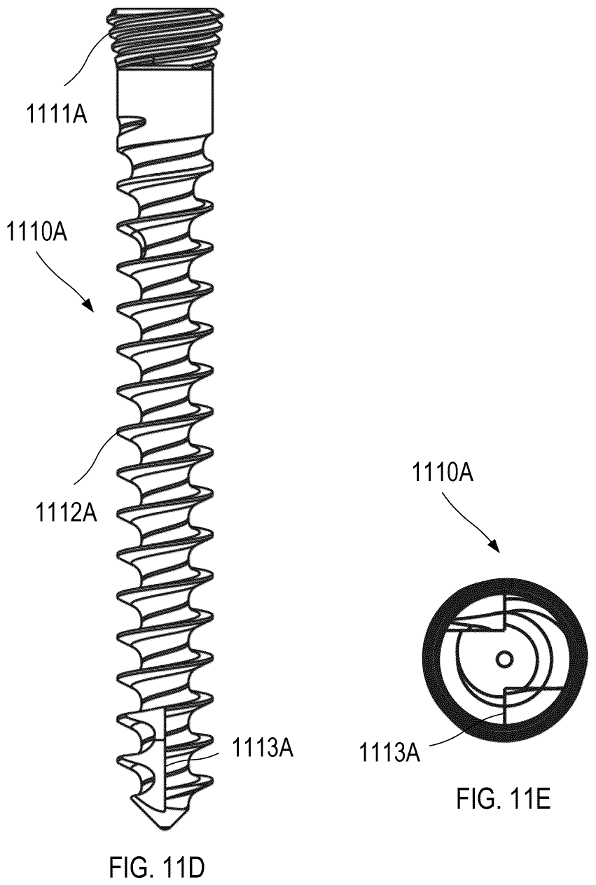

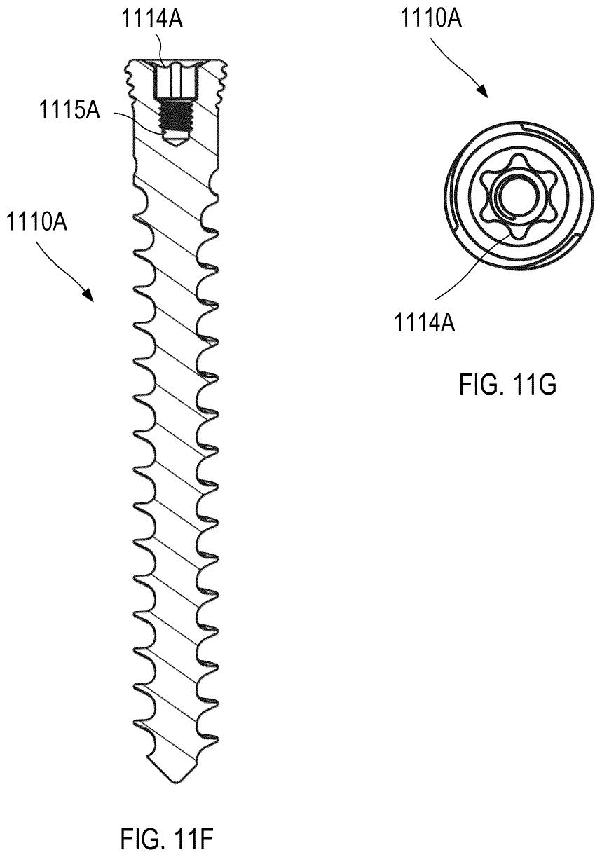

FIGS. 11A to 11C depict aspects of various plate screw configurations, according to embodiments of the present invention. As shown here, a plate screw 1110 can be configured to engage an interbody via a threaded mechanism 1115 (e.g. machine threads). In some cases, a plate screw can include bone threads 1120. In some cases, a plate screw can include a self-tapping mechanism 1125. In some cases, plate screw 1110 can be referred to as a plate fixation mechanism. FIGS. 11D and 11E depict aspects of a plate fixation mechanism 1110A, according to embodiments of the present invention. In some cases, plate fixation mechanism 1110A can be referred to as a plate screw. In some cases, plate fixation mechanism 1110A can be referred to as an iliac screw. As shown here, a plate fixation mechanism 1110A can include locking threads 1111A configured for fixation with an interarticular implant or interbody. Plate fixation mechanism 1110A can also include bone screw threads 1112A for fixation into patient tissue or bone, for example patient ilium bone. Plate fixation mechanism 1110A may also include a self-tapping feature 1113A. As depicted in FIGS. 11F and 11G, a plate fixation mechanism 1110A can include a driving feature 1114A, such as a T-25 hexalobular driving feature. In some cases, plate fixation mechanism 1110A can include a retaining feature 1115A for a driver.

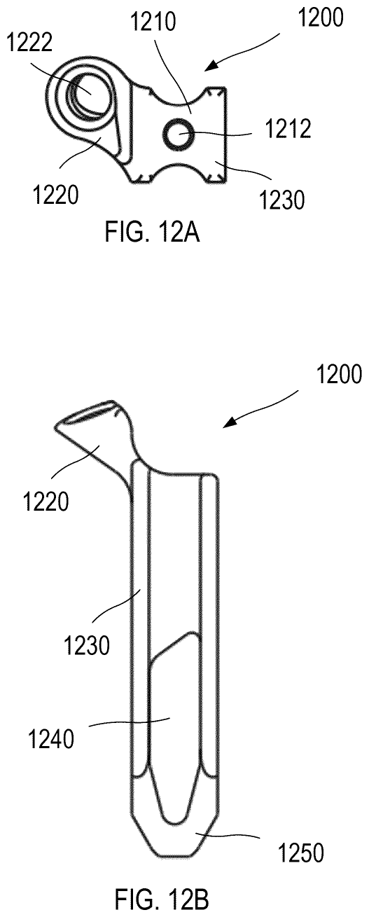

Embodiments of the present invention encompass a variety of interlocking configurations involving plates and plate screws. As shown in FIGS. 12A and 12B, an interbody 1200 can include a plate 1210 configured to engage a plate screw. For example, the plate 1210 can be configured with an overhanging plate portion 1220. The plate 1210 can include an aperture or through-hole 1212 configured to receive a pin, and the overhanging plate portion 1220 can include an aperture 1222 configured to receive a plate screw. In some embodiments, aperture 1222 is configured to receive or engage a sacral to iliac screw. In some cases, the plate 1210 can include an I-beam cross section portion 1230 configured to prevent or inhibit migration. In some cases, aperture 1212 can be part of a central cannula of the interbody. As shown in FIG. 12B, the overhanging plate 1220 can be at an angle, for example at an angle between about 30 degrees and about 90 degrees. An I-beam portion 1230 (or a section thereof) of the interbody can be a porous lattice structure. The interbody 1200 may include an interior section or opening 1240 that is configured to receive a graft material. In some cases, opening 1240 can be referred to as a central cannula. The interbody 1200 may include a tapered bottom or distal section 1250, for facilitating tapping into a joint.

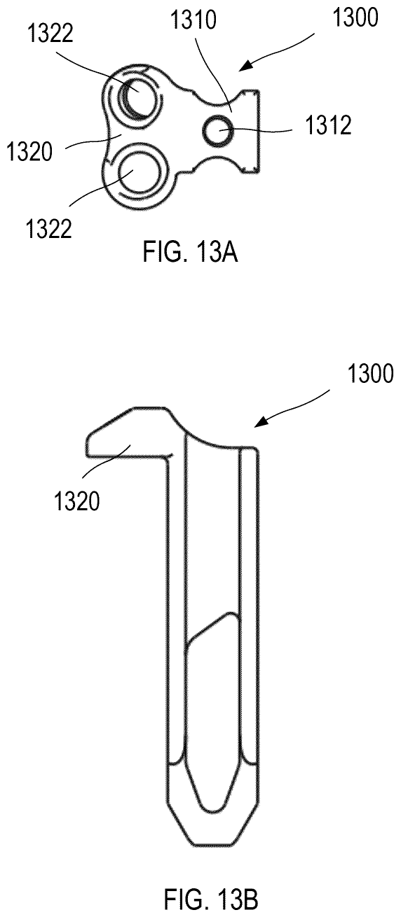

As shown in FIGS. 13A and 13B, an interbody 1300 can include a plate 1310 configured to engage one or more plate screws. For example, the plate 1310 can be configured with an overhanging plate portion 1320. The plate 1310 can include an aperture or through-hole 1312 configured to receive a pin, and the overhanging plate portion 1320 can include one or more apertures 1322 configured to receive the plate screw(s). In some embodiments, one aperture 1322 is configured to receive or engage a sacral to iliac screw and another aperture 1322 is configured to receive or engage a sacral screw. As shown in FIG. 13B, the overhanging plate 1320 can be at angle, for example at an angle of about 90 degrees.

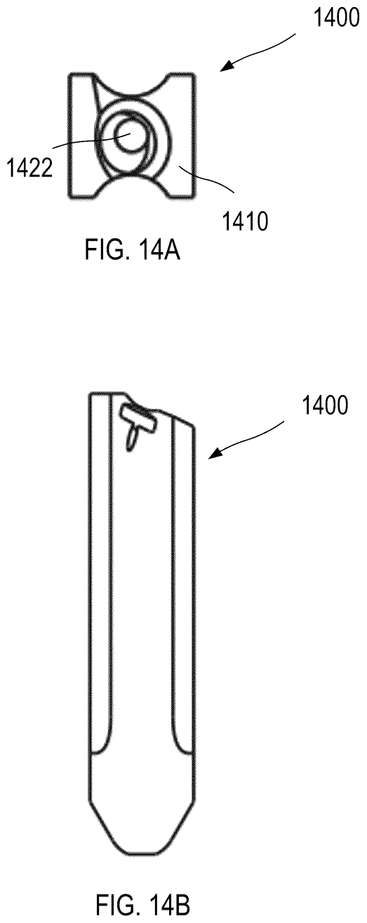

As shown in FIGS. 14A and 14B, an interbody 1400 can include a plate 1410 configured to engage a plate screw. For example, the plate 1410 can include an aperture 1422 configured to receive a plate screw. In some embodiments, aperture 1422 is configured to receive or engage a sacral to iliac screw. In the embodiment depicted in FIGS. 14A and 14B, the plate screw interlocks directly into the interbody, and there is no plate overhand.

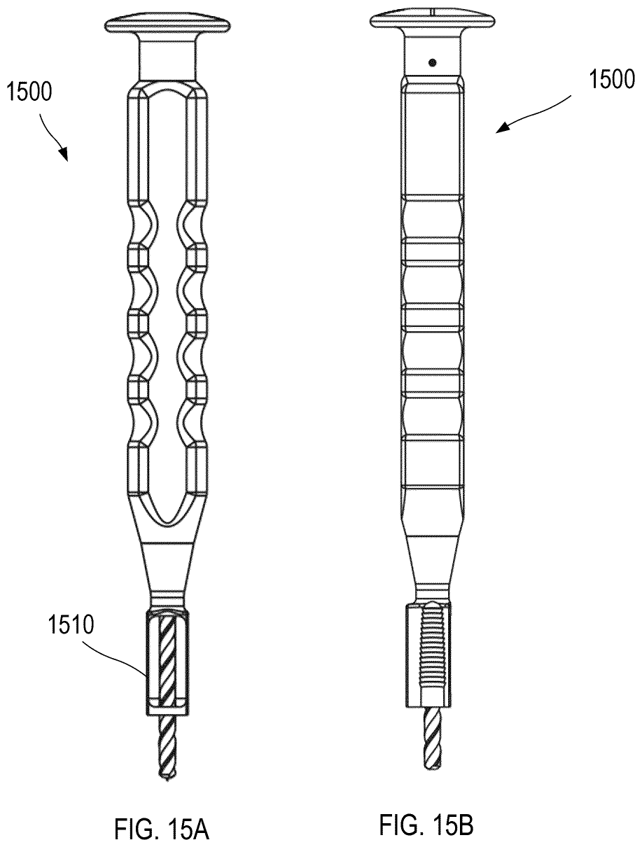

FIGS. 15A and 15B depict aspects of a broach or box cut device 1500, according to embodiments of the present invention. As discussed elsewhere herein, a surgeon or operator can use a ream device as a guide for a box cut device. A broach or box cut device 1500 can be provided, positioned, placed, and/or advanced over a ream device. In some embodiments, a surgeon or operator can use a broach or box cut device 1500 to create a channel or receptacle in tissue (e.g. bone) of a patient, and the channel or receptacle can receive or at least partially receive an interbody. As shown here, a broach or box cut device 1500 can include a window 1510 that operates to expel tissue such as bone, for example while impacting and/or collecting bone or bone graft. In some embodiments, a broach or box cut device 1500 can be used to remove sacral and/or iliac bone of a patient to create a receptacle for an implant body, as part of a sacroiliac fusion procedure.

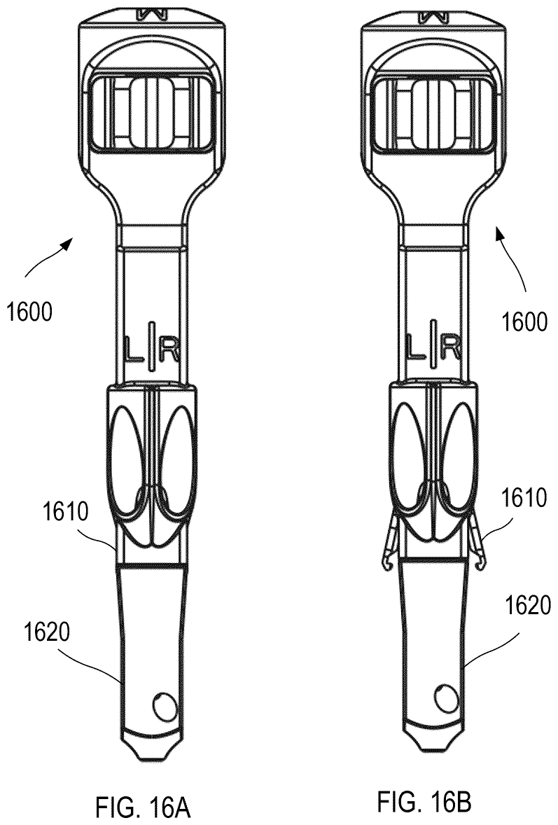

FIGS. 16A and 16B depict aspects of an inserter or impact tool 1600, according to embodiments of the present invention. As shown here, an inserter or impact tool 1600 can include one or more actuating arms that operate to hold and/or release an interbody 1620. In some embodiments, an inserter or impact tool 1600 can be used to impact an interbody device 1620 into a joint cavity. In some cases, an inserter or impact tool 1600 can be used to advance or force an interbody device 1620 into or at least partially into the sacroiliac joint space or cavity. In some cases, an inserter or impact tool 1620 can be used to advance or force an interbody device 1620 into or at least partially into a channel or receptacle that has been formed in the sacroiliac joint by a broach or box cut device. In some embodiments, an inserter or impact tool 1600 can be used to inserting an interbody or implant body in a receptacle that has been formed in tissue of a patient, for example, as part of a sacroiliac fusion procedure.

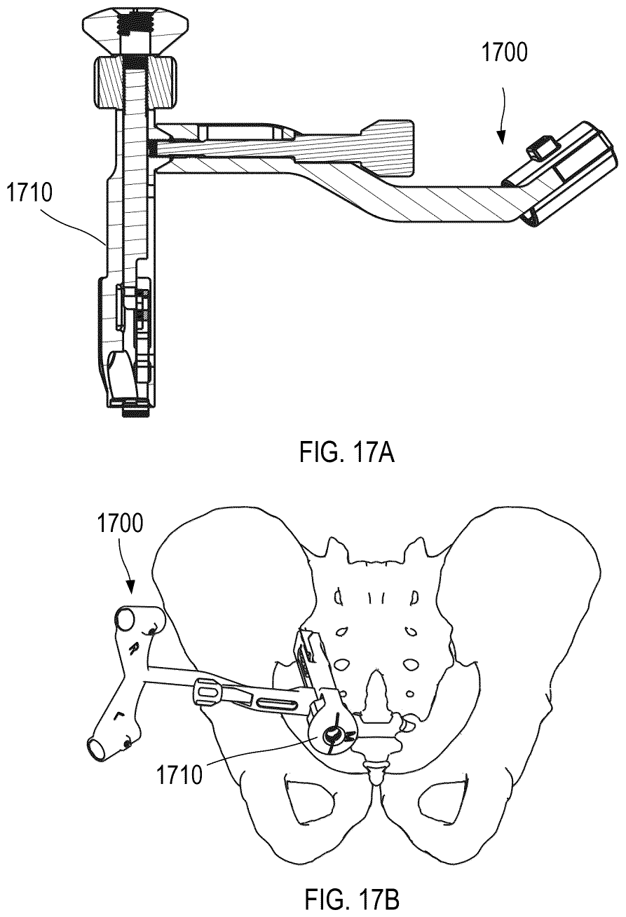

In some cases, an outrigger instrument can be used in conjunction with one or more tools such as those discussed elsewhere herein. For example, an outrigger instrument can be used to stabilize or hold one or more tools, as part of a sacroiliac fusion procedure. FIGS. 17A and 17B depict an outrigger instrument 1700 engaged with an inserter or impact tool 1710. A surgeon or operator can use the outrigger instrument 1700 to maintain the impact tool 1710 in a fixed position or at a fixed trajectory, as part of a sacroiliac fusion procedure.

Embodiments of the present invention are well suited for use in imaging procedures, and in particular embodiments, for imaging procedures that involve finding or locating a joint. Exemplary procedures may include placing a C arm at true 0 all axes, tilting the C arm around a transverse plane 15-30 degrees so that it is aligned to an anterior wall of sacrum, for an inlet view. Procedures may also involve moving the C arm along transverse plane lateral from midline (non-working side) to where the sacroiliac (SI) joints are superimposed. Accomplishing this can involve moving from a working to a non-working side and observing the separated anterior and posterior joint until they are superimposed about 20-35 degrees. This can be observed by living the C arm while it is rainbowed from a working side to a non-working side. Note at this point one should also be able to see the opening of the joint on the posterior side on the x-ray. Procedures may also involve placing a pin 5 mm above a posterior opening to the joint. Procedures may also include aligning a trajectory to be parallel with the C arm. Procedures can also include tapping a pin in a few millimeters.

Any of a variety of instruments and/or tools can be used to implant or remove one or more aspects of an implant assembly, according to embodiments of the present invention. Exemplary instruments and/or tools include broach devices, inserter devices, and outrigger devices.

Although the preceding description contains significant detail in relation to certain preferred embodiments, it should not be construed as limiting the scope of the invention but rather as providing illustrations of the preferred embodiments.

Embodiments of the present invention encompass kits having one or more components of a system as disclosed herein. In some embodiments, the kit includes one or more system components, along with instructions for using the component(s) for example according to any of the methods disclosed herein.

All features of the described systems and devices are applicable to the described methods mutatis mutandis, and vice versa.

In addition, each reference provided herein in incorporated by reference in its entirety to the same extent as if each reference were individually incorporated by reference. Relatedly, all publications, patents, patent applications, journal articles, books, technical references, and the like mentioned in this specification are herein incorporated by reference to the same extent as if each individual publication, patent, patent application, journal article, book, technical reference, or the like was specifically and individually indicated to be incorporated by reference.

While preferred embodiments of the present disclosure have been shown and described herein, it will be understood to those skilled in the art that such embodiments are provided by way of example only. Numerous variations, changes, and substitutions will now occur to those skilled in the art without departing from embodiments of the present invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A sacroiliac fusion system for treating a patient, comprising:
an implant body having a medial side configured to engage a sacrum of a patient, a lateral side configured to engage an ilium of the patient, a distal portion, and a proximal portion having an opening configured to receive a screw; and
a screw having a distal portion and proximal portion, wherein the distal portion comprises a threaded portion configured to engage bone of the patient and the proximal portion is configured engage the opening in the proximal portion of the implant body.

2. The system according to claim 1, wherein the screw engages both the ilium and the sacrum of the patient.

3. The system according to claim 1, wherein the screw is a first screw configured to engage the sacrum, and wherein the system further comprises a second screw configured to engage the ilium.

4. The system according to claim 1, wherein the proximal portion of the screw locks rigidly to the opening in the implant body.

5. The system according to claim 1, wherein the implant body is comprised of a porous structure configured for bony ingrowth.

6. The system according to claim 1, wherein the implant body has a window configured to contain bone graft.

7. The system according to claim 1, wherein the implant body has an hourglass shaped cross-section comprising a narrow section configured to be on the superior and inferior sides of the implant, and a narrowest point configured to be located at a sacroiliac joint of the patient.

8. The system according to claim 1, wherein the screw is a first screw, wherein the system further comprises a second screw configured to engage the ilium and the sacrum, and wherein the second screw is substantially perpendicular to a long axis of the implant body.

9. The system according to claim 8, wherein the second screw comprises a threaded portion configured to engage the implant body.

10. The system according to claim 8, wherein a head of the second screw compresses the ilium to the implant body.

11. The system according to claim 8, wherein a head of the second screw compresses the sacrum to the implant body.

12. The system according to claim 8, wherein the head of the second screw compresses the ilium to the sacrum.

13. The system according to claim 1, wherein the screw is a first screw, wherein the system further comprises a second screw configured to engage the ilium and the sacrum, and wherein the second screw engages a long axis of the implant body at an acute angle.

14. A method performing a sacroiliac fusion procedure on a patient, the method comprising:
placing a guide pin in a plane of a sacroiliac joint of the patient;
removing sacral and iliac bone of the patient to create a receptacle for an implant body;
inserting the implant body in the receptacle; and
securing the implant body to the patient with a screw.

15. The method according to claim 14, wherein the implant body comprises a medial side configured to engage a sacrum of a patient, a lateral side configured to engage an ilium of the patient, a distal portion, and a proximal portion having an opening configured to receive the screw, and
wherein the screw comprises a distal portion and proximal portion, wherein the distal portion comprises a threaded portion configured to engage bone of the patient and the proximal portion is configured engage the opening in the proximal portion of the implant body.

16. The method according to claim 15, wherein the screw engages both the ilium and the sacrum of the patient.

17. The method according to claim 15, wherein the screw is a first screw configured to engage the sacrum, and wherein the system further comprises a second screw configured to engage the ilium.

18. The method according to claim 15, wherein the proximal portion of the screw locks rigidly to the opening in the implant body.

19. The method according to claim 15, wherein the implant body is comprised of a porous structure configured for bony ingrowth.

20. The method according to claim 15, wherein the implant body has a window configured to contain bone graft.

21. The method according to claim 15, wherein the implant body has an hourglass shaped cross-section comprising a narrow section configured to be on the superior and inferior sides of the implant, and a narrowest point configured to be located at a sacroiliac joint of the patient.

22. The method according to claim 15, wherein the screw is a first screw, wherein the system further comprises a second screw configured to engage the ilium and the sacrum, and wherein the second screw is substantially perpendicular to a long axis of the implant body.

23. The method according to claim 22, wherein the second screw comprises a threaded portion configured to engage the implant body.

24. The method according to claim 22, wherein a head of the second screw compresses the ilium to the implant body.

25. The method according to claim 22, wherein a head of the second screw compresses the sacrum to the implant body.

26. The method according to claim 22, wherein the head of the second screw compresses the ilium to the sacrum.

27. The method according to claim 15, wherein the screw is a first screw, wherein the system further comprises a second screw configured to engage the ilium and the sacrum, and wherein the second screw engages a long axis of the implant body at an acute angle.

28. The method according to claim 14, wherein the step of removing sacral and iliac bone of the patient comprises using a box cut device, wherein the step of inserting the implant body in the receptacle comprises using an inserter device, or wherein the step of removing the sacral and iliac bone or inserting the implant body comprises using an outrigger device.

29. A sacroiliac fusion system, comprising:
an interbody; and
a screw.

30. The system according to claim 29, wherein the screw is a plate screw.
